(19) United States
(12) Patent Application Publication
(10) Pub. No.: US 2024/0238228 A1
(43) Pub. Date: Jul. 18, 2024

(54) COMPOSITIONS AND METHODS FOR TREATING ALLERGIES AND INFLAMMATORY CONDITIONS

(71) Applicant: UNIVERSITY OF PITTSBURGH-OF THE COMMONWEALTH SYSTEM OF HIGHER EDUCATION, Pittsburgh, PA (US)

(72) Inventors: Louis D. FALO, Jr., Bradfordwoods, PA (US); Emrullah KORKMAZ, Pittsburgh, PA (US); Tina L. SUMPTER, Murrysville, PA (US)

(21) Appl. No.: 18/559,403

(22) PCT Filed: May 6, 2022
(86) PCT No.: PCT/US2022/028151

§ 371 Date: Nov. 7, 2023

Related U.S. Application Data
(60) Provisional application No. 63/185,912, filed on May 7, 2021.

(51) Int. Cl. A61K 31/18; A61K 38/17; A61K 38/20; A61K 45/06; A61P 37/08

(52) U.S. Cl. CPC A61K 31/18; A61K 38/1709; A61K 38/2066; A61K 45/06; A61P 37/08

(57) ABSTRACT

Provided herein are compositions and methods of reducing inflammation in a subject comprising administering to the subject a pharmaceutically effective amount of a mast cell desensitizing composition.

### CROSS-REFERENCE TO RELATED APPLICATIONS

This application claims the benefit of U.S. Provisional Application No. 63/185,912, filed May 7, 2021, which is expressly incorporated herein by reference in its entirety.

### STATEMENT REGARDING FEDERALLY SPONSORED RESEARCH OR DEVELOPMENT

This invention was made with government support under grant numbers AR068249; AR071277 and AR067250 awarded by the National Institutes of Health. The government has certain rights in the invention.

### FIELD

The present disclosure relates to compositions and methods for treating allergies and inflammatory diseases.

### BACKGROUND

Mast cells (MCs) initiate and amplify immune responses in vascularized tissues, including the skin. Skin-resident MCs are primed with FcεRI-bound IgE in the steady state and poised to respond to polyvalent antigen (Ag). Upon Ag exposure, signaling initiated by the FcεRI induces immediate release of preformed, granulated proteases and cytokines and lipid mediators via exocytosis dependent and independent mechanisms. Downstream STAT5 and NFκB activation promote synthesis of inflammatory and Th2-promoting cytokines, including TNF and IL-13. Cumulatively, this response initiates anti-helminth and anti-bacterial immune responses and perpetuates allergic inflammation.

Dermal MCs reside in close proximity to CGRP⁺ or PGP-5⁺ sensory nerve fibers. Within the MC-neuron synapse bidirectional communication skews the outcome of local IgE-initiated immune responses. In this regard, sensory nerve fibers release neuropeptides including substance P (SP), Calcitonin Gene-Related Peptide (CGRP) and neurokinin A (NKA). While SP and CGRP have been extensively studied in MCs and in the skin, little is known about the immune functions of NKA.

Neurokinin A is transcribed from the Tac1 gene and interacts with the NK2R, a G-protein coupled receptor (GPCR). NKA and the NK2R have been reported to have pro- and anti-inflammatory effects. In mucosal tissues, NK2R signaling is associated with inflammation. But in the skin, NK2R antagonism enhances allergic contact hypersensitivity (CHS) and NKA administration blocks CHS. Specific to MCs, NKA induces histamine release from mucosal MCs from the lung but not connective tissue MCs from the skin, the heart, or peritoneal MCs suggesting tissue-specific roles for NKA/NK2R in MC activation.

What are needed are effective therapies for inflammation and/or type I hypersensitivity reactions that target or modulate mast cells and/or mast cell components.

### SUMMARY

Herein, the hypothesis that NKA affects IgE-initiated MC function in the skin with the murine passive cutaneous anaphylaxis (PCA) model and in vitro using bone marrow (BM)MCs and peritoneal (P)MCs is tested. That data show that administration of NKA prior to PCA induction reduced degranulation-associated edema and pro-inflammatory cytokine levels in vivo in an IL-10 dependent manner. Similarly, NKA inhibited MC activation in an IL-10 dependent manner in vitro. It is shown herein that both BMMCs and PMCs released the IL-10-degrading protease, calpain, through the glyburide-sensitive ABCA1 and that this pathway is inhibited by NKA. In turn, NKA inhibited IL-10 degradation, thereby prolonging the availability of IL-10 in the microenvironment. Importantly, it is demonstrated herein that bypassing NKA and directly targeting the ABCA1 is sufficient to inhibit MC activation in vitro and in vivo.

Accordingly, provided herein are methods of treating an inflammation in a subject and methods of treating a type I hypersensitivity reaction in a subject comprising administering to the subject a pharmaceutically effective amount of a mast cell desensitizing composition.

In some embodiments, the type I hypersensitivity reaction is selected from the group consisting of asthma, rhinitis, conjunctivitis, and dermatitis.

In some embodiments, the dermatitis is psoriasis, eczema or seborrheic dermatitis.

In some embodiments, the eczema is atopic dermatitis.

In some embodiments, the type I hypersensitivity reaction is selected from the group consisting of anaphylaxis, urticaria, and angioedema. In some embodiments, the method further comprises administering one or more type I hypersensitivity reaction antigens to the subject.

In some embodiments, the one or more type I hypersensitivity reaction antigens are administered to a same cutaneous microenvironment as the mast cell desensitizing composition.

In some embodiments, the type I hypersensitivity reaction is a food allergy or a drug allergy.

In some embodiments, the type I hypersensitivity reaction is a food allergy and wherein the method further comprises administering to the subject an antigen of the food.

In some embodiments, the food is a peanut.

In some embodiments, the mast cell is a connective tissue mast cell.

In some embodiments, the mast cell is a mucosal mast cell.

In some embodiments, the mast cell desensitizing composition comprises an ABCA1 inhibitor.

In some embodiments, the ABCA1 inhibitor is selected from the group of a glyburide, a probucol, a tocofersolan and a Vitamin E. In some embodiments, the ABCA1 inhibitor is a glyburide.

In some embodiments, the mast cell desensitizing composition comprises a neurokinin A.

In some embodiments, the neurokinin A has a sequence of HKTDSFVGLM (SEQ ID NO:2) or a functional fragment thereof. In some embodiments, the mast cell desensitizing composition is botulinum toxin A.

In some embodiments, the method further comprises administering to the subject an IL-10.

In some embodiments, the administration is intradermal or transdermal.

In some embodiments, the administration is via one or more microneedles.

In some embodiments, the one or more microneedles are dissolvable.

### DESCRIPTION OF DRAWINGS

FIG. 1(A-G) shows that NKA inhibits the early and late phases of PCA. The ears of WT (C57BL/6) mice were injected with vehicle or IgE on d0. On dl, mice were injected (i.v.) with cross-linking Ag. (A) Twenty-four h following Ag, mice were euthanized and NKA was detected in the tissue 24 hours after Ag. Data show the mean+1 SEM from 2 independent experiments with 2-4 mice each. (B) The expression of the NK2R was evaluated in skin sections by immunofluorescent microscopy (C-G) Mice were pre-treated with vehicle (PBS) or NKA (10 mg, i.d) 3 hours prior to the administration of crosslinking Ag. (C) The change in ear thickness at the indicated time after Ag compared to d0. Data are the mean±SEM from 12-15 mice from 2-3 independent experiments with 4-6 mice each. (D, F) Microscopic images from skin sections, 2 h and 24 hours after PCA induction. Images are representative of 3 mice per group. H&E, 200×(E) Evans blue dye extracted from the tissue 90 min following cross-linking Ag, 3 independent experiments with 3 mice per group each. (G) Semi-quantitative expression of cytokines in skin homogenates. Heat map depicts the relative expression of proteins normalized to the vehicle control from 3 independent experiments. Table indicates statistical significance comparing IgE-treated skin with skin pretreated with NKA (IgE+NKA). p-values were determined by 2-way ANOVA, with Bonferroni post-hoc analysis or by paired t-test.

FIG. 2(A-F) shows that NKA inhibits the release of STAT5 transcribed cytokines. BMMCs were loaded with IgE (1.0 mg/mL) then activated with cross-linking Ag (DNP-HSA 100 ng/mL) with and without NKA (as indicated or 1000 nM) (A) IL-13 and TNF levels in cell free supernatants collected 22-24 hours after activation. (B) IL13 or TNF RNA from cells activated for 60 min. Data show the mean expression three independent experiments. (C-D) pTyr694-STAT5 was detected by flow cytometry following activation with Ag (top panels) with or without NKA (bottom panels) for the indicated times. (C) shows representative flow plots, numbers indicate the Mean Fluorescent Intensity (MFI), shaded histogram shows pTry694-STAT5 from unstimulated cells (D) summarizes the MFI for pTyr694-STAT5 from three independent experiments. (E-F) Nuclear localization of STAT5B evaluated by ImageStream. (E) Representative analysis from 5 cells following 60 min of activation in the presence or absence of NKA. (F) Mean+1 SEM for the Similarity Scores from three independent experiments. p-values were determined by 2-way ANOVA, with Bonferroni post-hoc analysis or by paired t-test.

FIG. 3(A-I) shows that IL-10 is required for NKA to suppress FceRI-initiated MC activation in vitro and in vivo. (A-D) MCs were activated as in FIG. 2. (A) IL-10 by ELISA from supernatants collected 20-24 h later (B) IL10 RNA from cells activated for 60 min. (C-D) Cytokines detected in supernatants from (C) BMMCs or (D) PMCs collected 20-24 h after activation (E) Vehicle control or NKA was injected into either MC-deficient (Mcpt5 Cre⁺×Rosa26 DTA⁺) or MC-sufficient mice (Mcpt5 Cre⁻×Rosa26 DTA⁺) and IL10 expression was evaluated 3 hours later. Data show 2 experiments with 2-3 mice per group. (F-H) PCA was induced in Mcpt5 Cre⁺×IL10^{WT} and Mcpt5Cre⁺×IL10^{fl/fl} mice. (F) shows ear measurements at 2 and 24 hours and (H) shows cytokines detected in the skin by ELISA at 24 h. (I) Neurokinin A in skin homogenates from WT B6 skin, IL-10 KO mice or B6 mice treated with IL-10 (1.0 ng/ear) at the time of IgE injection was detected by ELISA 24 h after PCA. Data are the mean±SEM from 2 independent experiments using 2-3 mice per group. For the NKA ELISA, mean±SEM from 2-3 mice per experiment, 2 independent experiments for B6 and IL10 KO, 3 mice from 1 experiment for B6+IL10. p-values were determined by 2-way ANOVA, with Bonferroni post-hoc analysis or by paired t-test.

FIG. 4(A-H) shows that NKA changes the MC secretome. (A-D) IgE-loaded MCs were activated with cross-linking Ag (IgE+Ag) or cultured with vehicle (control) for 30 minutes. Representative flow plots depicting LAMP-1 and avidin staining are depicted for (A) BMMCs and (B) PMCs (C, F) summarized the mean percentage positive ±1 SEM from 3 independent experiments. (E-H) BMMCs were activated for 60 minutes. Supernatants were analyzed by MS/MS. Venn diagram depicts the number of unique and overlapping peptides detected. (F) Pathway analysis was done with Panther (G) Semiquantitative representation of the coverage area from three independent runs (H) Quantitation from three independent experiments. p-values were determined by 2-way ANOVA with Bonferroni post-hoc analysis.

FIG. 5(A-H) shows that NKA inhibits the release of the IL-10 degrading enzyme, calpain. (A-B) Calpain activity was evaluated in (A) BMMCs or (B) PMCs activated with IgE+Ag with or without NKA for 60 min. (C-D) Recombinant (r) calpain was incubated for 60 minutes with rIL-10. IL-10 was detected by Western Blot. (C) shows a representative blot and (D) summarizes the percentage of IL-10 degradation, calculated by normalizing the densitometry of IL-10+calpain to IL-10 alone from three independent experiments. (E) Cell free supernatants from IgE-activated BMMCs were collected at the indicated time then assayed for IL-10 and for calpain activity. (F) Supernatants were collected 10 minutes after activation, then pulsed with Ac-cal (20 mg/mL) for 30 min prior to assaying IL-10 by ELISA. The amount of IL-10 recovered from 2-3 experiments is shown. (G) IL-10 recovered 60 min after activation of BMMCs from 3 independent experiments. (H) Supernatants were collected 30 min after activation then pulsed with rIL-10 for 30 minutes. The amount of IL-10 recovered by ELISA was normalized to the amount of IL-10 recovered from media pulsed with IL-10. Data depict the mean±SEM from 3 independent experiments but where indicated. p-values were determined by 2-way ANOVA with Bonferroni post-hoc analysis.

FIG. 6(A-H) shows the inhibition of the ABCA1 efflux channel is redundant with the effects of NKA in vitro and in vivo. (A-B) Expression of the ABCA1 on the surface of BMMCs or PMCs 30 minutes after IgE-initiated activation in the presence of vehicle control (PBS) or NKA (1000 nM). (A) depicts a representative flow plot, values denotes the percentage of ABCA1⁺ cells and (B) summarizes the mean±SEM of the percentage of ABCA1⁺ cells following IgE-initiated activation from 3 independent experiments. (C) BMMCs were treated with the vehicle control (“0”, 0.0125% DMSO) or glyburide for 10 min prior to activation. Supernatants were collected 30 min after activation and assayed for calpain activity and IL-10. (D) BMMCs or PMCs were pretreated with vehicle or glyburide (50 mM) then IgE-activated for 30 minutes and stained with anti-LAMP-1 or avidin. Graph depicts the mean±SEM of the percentage of positive cells. (E) BMMCs or PMCs were IgE-activated and cultured 20-24 hours in the presence of vehicle control (0.0125% DMSO) or glyburide (as indicated for BMMCs, 50 mM for PMCs) and NKA (1000 nM). Supernatants were assayed by ELISA. (F-H) PCA was induced in mice that were pretreated with glyburide (2.5 mg/kg) 3 hours prior to induction. The effects of glyburide were measured as a function of (F) ear thickness, (G) extravasation of Evans blue dye, or (H) cytokines detected in the skin 24 hours following induction of PCA. In vitro data summarize the mean±SEM from 3 independents. In vivo data show 2 independent experiments, each with 2-3 mice per group. p-values were determined by 2-way ANOVA with Bonferroni post-hoc analysis or by paired t-test.

FIG. 7(A-D) shows that MCs are poised to respond to neurokinin A in vivo. (A) Expression of the NK2R on naïve cutaneous MCs as detected by flow cytometry. Values on histogram are the mean percentage positive +1 SD from 6 mice (three independent experiments, with 2 mice each). (B) Myeloperoxidase activity in skin homogenates was detected 24 hours after induction of PCA. Data are from 3 experiments with 2-3 mice per group. (C) Cytokine arrays were performed on protein isolated from mouse skin 24 hours after the induction of PCA with or without NKA. Positive control spots are in the squares. (D) shows a representative set of arrays. Bars show the mean+1 SEM of the relative densitometry for the indicated cytokine normalized to positive controls from three independent experiments. *denotes p<0.05 by paired t-test.

FIG. 8(A-C) shows that in vitro differentiated MCs express the NK2R. (A) The purity and NK2R expression on BMMCs (left) and PMCs (right) is shown. Percentages are the mean±SD from a minimum of three experiments. (B-C) β-hexosaminidase release from BMMCs (B) or PMCs (C) activated with IgE and Ag (100 ng/mL DNP-HAS or as indicated) in the presence of NKA (1000 nM). Bars present the mean percentage release ±1 SEM from 3 independent experiments.

FIG. 9(A-C) shows that Neurokinin A induces IL-10-GFP expression in mast cells. Induction of IL-10-GFP was evaluated in VERT-X mice pretreated with NKA or vehicle control prior to the induction of PCA. (A) depicts the gating strategy used to identify MCs in skin homogenates. Top panels depict a VERT-X mouse, bottom panels show a MC-deficient Mcpt5Cre⁺×Rosa26DTA⁺ mouse. (B) Representative plots showing IL-10-GFP⁺ MCs. Numbers indicated the percentage positive. (C) Summarizes the percentage of IL-10-GFP⁺ MCs from three independent experiments, with 1-2 mice per group. A naïve control was included in each experiment.

FIG. 10(A-C) shows glyburide reduces cutaneous inflammation in a murine model of atopic dermatitis. Atopic dermatitis was induced by treating ears with MC903 (4 nmol/ear, daily). The contralateral ear was treated with the vehicle for MC903. Mice were treated with either glyburide (2.5 mg/kg, i.p., daily) or vehicle control. (A) Ear thickness was measured daily. (B-C) On day 11, mice were euthanized and ear tissue embedded sectioned and stained with (B) H & E to evaluate cellular infiltration or (C) Avidin (AvRho) to evaluate mast cells. (A) shows the mean+/−SEM from 2 experiments with 3-4 mice per group. (B-C) show representative images from 3-7 mice over two experiments.

FIG. 11(A-C) shows glyburide and peanut antigen delivered by to the skin microenvironment by microneedle arrays desensitizes allergy to peanut allergy. Mice were sensitized to epicutaneously with complete peanut extract (CPE, 100 mg per mouse, weekly for six weeks. Sensitization was confirmed by challenge with CPE (i.p, 10 mg/mouse). Sensitized mice were treated with microneedle arrays loaded with purified peanut extract (PE) or glyburide with PE two times at one week intervals. One week later, mice were challenged with CPE. (A) Anaphylaxis was scored with a standard scale by two independent researchers 40 min after challenge. (B) the change in temperature was calculated by subtracting Tm immediately prior to challenge from the Tm 60 min after challenge. (C) Total serum IgE was evaluated by ELISA. Data depict 3-4 mice per group from one experiment.

FIG. 12(A-D) shows botulinum toxin A (BOTOX) delivered with peanut antigen by microneedle arrays reduces IL-33 expression and desensitizes allergy to peanut allergen. (A) MNAs loaded with peanut extract or peanut extract and BOTOX were applied to mouse skin. 12 h later, mice were euthanized, RNA extracted and expression of IL33 determined by qRT-PCR. (B-D) Mice were sensitized epicutaneously with complete peanut extract (six times at one week intervals). Sensitization was confirmed by CPE challenge. One week later MNAs were applied. One week after MNA application, mice were challenged with CPE. (B) depicts the change in temperature 60 min after challenge. (C-D) Peanut specific antibodies were detected in serum by ELISA.

### DETAILED DESCRIPTION

Disclosed herein are compositions and methods for preventing or reducing an inflammation in a subject comprising administering to the subject a therapeutically effective amount of a mast cell desensitizing composition. In some embodiments, the administration treats a type I hypersensitivity reaction in the subject.

Terms used throughout this application are to be construed with ordinary and typical meaning to those of ordinary skill in the art. However, Applicant desires that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of 20%, ±10%, +5%, or +1% from the measurable value.

“Activate”, “activating”, and “activation” mean to increase an activity, response, condition, or other biological parameter. This may also include, for example, a 10% increase in the activity, response, “or condition, as compared to the native or control level. Thus, the increase can be a 10, 20, 30, 40, 50, 60, 70, 80, 90, 100%, or any amount of reduction in between as compared to native or control levels.

“Administration” to a subject includes any route of introducing or delivering to a subject an agent (e.g., a mast cell desensitizing composition). Administration can be carried out by any suitable route, including oral, topical, intravenous, cutaneous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. In some embodiments, the compositions described here are administered intradermally or transdermally. In some embodiments, the compositions described here are administered intradermally or transdermally via microneedle or microneedle array. In some embodiments, the microneedle(s) is dissolvable. In some embodiments, the microneedle(s) used in the present invention is selected from those described in one or more of U.S. Pat. No. 8,834,423, PCT Publication No. WO 2017/120322, U.S. Publication No. 2018/0304062, U.S. Publication No. 2020/0353235, and PCT Publication No. WO 2021/178879. Administration includes self-administration and the administration by another.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property of the sequence from which it is derived such as a mast cell desensitizing property.

The terms “identity” “identical to” and “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. Neither N- nor C-terminal extensions nor insertions shall be construed as reducing identity or homology. A polynucleotide or polynucleotide region (or a polypeptide or polypeptide region) that has a certain percentage (for example, 80%, 85%, 90%, or 95%) of “sequence identity” to another sequence means that, when aligned over their full lengths, that percentage of bases (or amino acids) are the same in comparing the two sequences. This alignment and the percent homology or sequence identity can be determined using software programs known in the art. In one embodiment, default parameters are used for alignment. In one embodiment a BLAST program is used with default parameters. In one embodiment, BLAST programs BLASTN and BLASTP are used with the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, “inflammation” refers to one or more of immune cell infiltration, leukocyte infiltration, capillary dilation, redness, heat and pain in an area of a subject.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, a “mast cell desensitizing composition” refers to a composition that reduces mast cell activation initiated by FcεR1 and/or reduces mast cell production or release of mediator compositions. Mast cell mediator compositions include, but are not limited to, calpain, histamine, β-hexosaminidase, chymases, tryptases, carboxypeptidase A and cytokines. In some embodiments, a mast cell desensitizing composition reduces mast cell production or release of calpain 1. In some embodiments, a mast cell desensitizing composition reduces activity or functionality of mast cell ABAC1 polypeptide. In some embodiments, a mast cell desensitizing composition comprises Neurokinin A. In some embodiments, a mast cell desensitizing composition comprises glyburide. In some embodiments, a mast cell desensitizing composition comprises botulinum toxin.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

As used herein, the term “reducing”, “reduce”, and other grammatical variations thereof generally means a decrease by a statistically significant amount. However, for avoidance of doubt, “reducing”, “reduce”, or “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Therapeutically effective amount” or “therapeutically effective dose” of a composition (e.g. a composition comprising a mast cell desensitizing composition) refers to an amount that is effective to achieve a desired therapeutic result. In some embodiments, a desired therapeutic result is a prevention or reduction of an inflammation in a subject. In some embodiments, a desired therapeutic result is a prevention or reduction of a type I hypersensitivity reaction in a subject. In some embodiments, a desired therapeutic result is systemic tolerance to the one or more type I hypersensitivity reaction antigens. In some embodiments, a desired therapeutic result is a prevention or reduction of asthma, rhinitis, conjunctivitis, or dermatitis in a subject. In some embodiments, a desired therapeutic result is a prevention or reduction of anaphylaxis, urticaria, angioedema, food allergy, or drug allergy in a subject. Therapeutically effective amounts of a composition comprising a mast cell desensitizing composition will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as mitigation of a cancer. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of a type I hypersensitivity reaction), during early onset (e.g., upon initial signs and symptoms of a type I hypersensitivity reaction), or after an established development of a type I hypersensitivity reaction. Prophylactic administration can occur for several days to years prior to the manifestation of symptoms of a disease (e.g., a type I hypersensitivity reaction).

“Type I hypersensitivity reaction” refers herein to an immune reaction that involves or is mediated by IgE bound to IgE receptors on mast cells. Antigen binding to the bound IgE results in the cross-linking of IgE on the mast cell surfaces and causes cellular degranulation and release of mast cell mediator compositions. The antigen that binds to IgE and initiates or causes the type I hypersensitivity reaction is referred to herein as a “type I hypersensitivity reaction antigen.”

### Compositions and Methods

Disclosed herein are compositions and methods for preventing or reducing an inflammation in a subject comprising administering to the subject a therapeutically effective amount of a mast cell desensitizing composition. In some embodiments, the administration treats a type I hypersensitivity reaction in the subject. A type I hypersensitivity reaction is an immune reaction that involves or is mediated by IgE bound to IgE receptors on mast cells. Mast cells include connective tissue mast cells and mucosal mast cells. In some embodiments, the mast cell is a connective tissue mast cell. In other embodiments, the mast is a mucosal mast cell. In some embodiments, the antigen that binds to IgE and initiates or causes the type I hypersensitivity reaction, referred to herein as a “type I hypersensitivity reaction antigen,” is administered to the subject in addition to the mast cell desensitizing composition. In some embodiments, more than one type I hypersensitivity reaction antigen, or in other words, different type I hypersensitivity reaction antigens, are administered to the subject. The one or more type I hypersensitivity reaction antigens can be administered before, concurrently or after the mast cell desensitizing composition. In some embodiments, the one or more type I hypersensitivity reaction antigens is administered to the same cutaneous microenvironment as the mast cell desensitizing composition. As used herein, “same cutaneous microenvironment” refers to an area on the subject that is within less than about 12 inches, about 11 inches, about 10 inches, about 9 inches, about 8 inches, about 7 inches, about 6 inches, about 5 inches, about 4 inches, about 3 inches, about 2 inches, about 1 inch, about 0.5 inch, about 0.25 inch, about 0.1 inch, or about 0.001 inch of an area within the first site of administration.

In some aspects, the type I hypersensitivity reaction is selected from the group consisting of asthma, rhinitis, conjunctivitis, and dermatitis. Accordingly, included herein is a treatment for asthma in a subject comprising administering to the subject a therapeutically effective amount of a mast cell desensitizing composition.

Also included is a treatment for a dermatitis in a subject comprising administering to the subject a therapeutically effective amount of a mast cell desensitizing composition. In some embodiments, the dermatitis is a psoriasis or an eczema. In some embodiments, the eczema is atopic dermatitis. In some embodiments, the dermatitis is a contact dermatitis, a diaper dermatitis, a dyshidrotic dermatitis, a neurodermatitis, a nummular dermatitis, a perioral dermatitis or a seborrheic dermatitis.

Further included is a treatment for rhinitis in a subject comprising administering to the subject a therapeutically effective amount of a mast cell desensitizing composition. Still further included is a treatment for conjunctivitis in a subject comprising administering to the subject a therapeutically effective amount of a mast cell desensitizing composition.

In other or further embodiments, the type I hypersensitivity reaction is selected from the group consisting of anaphylaxis, urticaria, and angioedema. In some embodiments, the type I hypersensitivity reaction is a food allergy. The food allergy includes, but is not limited to, celery allergy, wheat allergy, crustacean allergy, egg allergy, fish allergy, lupin allergy, milk and dairy allergy, mollusc (such as mussels and oysters) allergy, mustard allergy, peanut allergy, tree nut allergy, sesame allergy, and soybean allergy. In some embodiments, the food allergy is a peanut allergy. In some embodiments, type I hypersensitivity reaction is a drug allergy. The present invention includes aspects where the type I hypersensitivity reaction is a food allergy and wherein the method further comprises administering to the subject an antigen of the food. The food antigen can be administered before, concurrently or after the mast cell desensitizing composition.

A mast cell desensitizing composition administered to a subject according to the present methods is a composition that reduces mast cell activation initiated by FcεR1 and/or reduces mast cell production or release of mediator compositions. In some embodiments, the mast cell desensitizing composition comprises an ABCA1 inhibitor. The term “ABCA1” refers herein to a polypeptide that is also referred to as ATP Binding Cassette Subfamily A Member 1, and in humans, is encoded by the ABCA1 gene. In some embodiments, the ABCA1 polypeptide is that identified in one or more publicly available databases as follows: HGNC: 29, Entrez Gene: 19, Ensembl: ENSG00000165029, OMIM: 600046, UniProtKB: 095477. In some embodiments, the ABCA1 polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. The ABCA1 polypeptide of SEQ ID NO: 1 may represent an immature or pre-processed form of mature ABCA1 polypeptide, and accordingly, included herein are mature or processed portions of the ABCA1 polypeptide in SEQ ID NO: 1.

As used herein, “ABAC1 inhibitor” refers to a composition that reduces activity or functionality of a mast cell ABAC1 polypeptide. In some embodiments, the ABCA1 inhibitor is selected from the group consisting of a glyburide, a probucol, a tocofersolan and a Vitamin E. In some embodiments, the ABCA1 inhibitor is glyburide (also known as glibenclamide) or a pharmaceutically acceptable salt, prodrug, or derivative thereof. In some embodiments, the glyburide has a structure below:

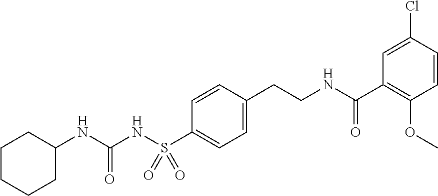 [SMILES: COc1ccc(Cl)cc1C(=O)NCCc1ccc(S(=O)(=O)NC(=O)NC2CCCCC2)cc1]

In some embodiments, the ABCA1 inhibitor is probucol or a pharmaceutically acceptable salt, prodrug, or derivative thereof. In some embodiments, the probucol has a structure below:

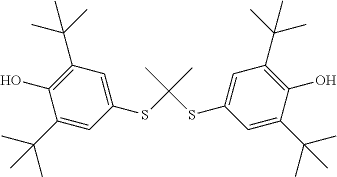 [SMILES: CC(C)(Sc1cc(C(C)(C)C)c(O)c(C(C)(C)C)c1)Sc1cc(C(C)(C)C)c(O)c(C(C)(C)C)c1]

In some embodiments, the ABCA1 inhibitor is tocofersolan or a pharmaceutically acceptable salt, prodrug, or derivative thereof. In some embodiments, the tocofersolan has a structure below:

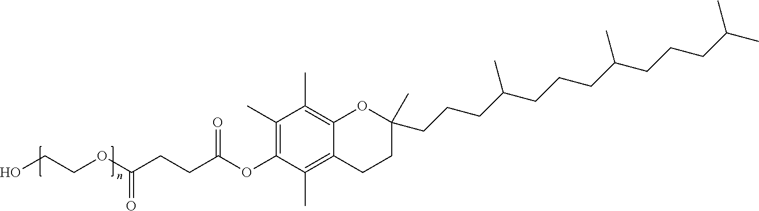

In some embodiments, the mast cell desensitizing composition comprises a neurokinin A. In some aspects, neurokinin A is a peptide comprising an amino acid sequence of HKTDSFVGLM (SEQ ID NO: 2), a functional fragment thereof, or a peptide having at or greater than about 80%, about 85%, about 90%, about 95%, or about 98% homology with SEQ ID NO: 2. In some embodiments, neurokinin A is a peptide consisting of an amino acid sequence of HKTDSFVGLM (SEQ ID NO: 2).

In some embodiments, the mast cell desensitizing composition comprises a botulinum toxin A. Botulinum toxin A includes those compositions referred to as “botulinum type A” or “botulinum toxin type A” in U.S. Publication No. 2007/0026019, U.S. Publication No. 2021/0024913, U.S. Pat. Nos. 8,501,196, 9,629,904, or U.S. Pat. No. 10,064,921, and the patent and journal references cited therein. These materials, including dosage ranges listed therein, are incorporated herein by reference.

In some embodiments, the mast cell desensitizing composition comprises a mast cell calpain 1 inhibitor. The term “calpain” refers herein to a polypeptide that is also referred to as Calcium-Activated Neutral Proteinase 1, and in humans, is encoded by the CAPN1 gene. In some embodiments, the calpain 1 polypeptide is that identified in one or more publicly available databases as follows: HGNC: 1476, Entrez Gene: 823, Ensembl: ENSG0000014216, OMIM: 114220, UniProtKB: P07384. In some embodiments, the calpain 1 polypeptide comprises the sequence of SEQ ID NO: 3, or a polypeptide sequence having at or greater than about 80%, about 85%, about 90%, about 95%, or about 98% homology with SEQ ID NO: 3, or a polypeptide comprising a portion of SEQ ID NO: 3. The calpain 1 polypeptide of SEQ ID NO: 3 may represent an immature or pre-processed form of mature calpain 1 polypeptide, and accordingly, included herein are mature or processed portions of the calpain 1 polypeptide in SEQ ID NO: 3. As used herein, “calpain 1 inhibitor” refers to a composition that reduces activity or functionality of a mast cell calpain 1 polypeptide.

As the timing of an inflammatory or type I hypersensitivity reaction can often not be predicted, it should be understood the disclosed methods of treating, preventing, reducing, and/or inhibiting the disease or disorder described herein can be used prior to or following the onset of the disease or disorder, to treat, prevent, inhibit, and/or reduce the disease or disorder or symptoms thereof. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3.2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of the disease or disorder; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48.60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months, 2, 3, 4, 5, 6, 7, 8, 9, 10, 15, 20, 25, 30, 35, 40, 45, 50, 55, 60 or more years after onset of the disease or disorder.

Dosing frequency for the compositions of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 minutes, once every 30 minutes, once every 20 minutes, or once every 10 minutes. Administration can also be continuous and adjusted to maintaining a level of the compound within any desired and specified range.

In some embodiments, one or more cytokines are administered to the subject before, concurrently, or after the mast cell desensitizing composition. In some aspects, the cytokine is an IL-10. The term “IL-10” refers herein to a polypeptide that is also referred to as Cytokine Synthesis Inhibitory Factor or CSIF, and in humans, is encoded by the IL10 gene. In some embodiments, the IL-10 polypeptide is that identified in one or more publicly available databases as follows: HGNC: 5962, Entrez Gene: 3586, Ensembl: ENSG00000136634, OMIM: 124092, UniProtKB: P22301. In some embodiments, the IL-10 polypeptide comprises the sequence of SEQ ID NO: 4, or a polypeptide sequence having at or greater than about 80%, about 85%, about 90%, about 95%, or about 98% homology with SEQ ID NO: 4, or a polypeptide comprising a portion of SEQ ID NO: 4. The IL-10 polypeptide of SEQ ID NO: 4 may represent an immature or pre-processed form of mature IL-10 polypeptide, and accordingly, included herein are mature or processed portions of the IL-10 polypeptide in SEQ ID NO: 4. Also included herein are kits comprising a mast cell desensitizing composition and IL-10.

As discussed above, “therapeutically effective amount” or “therapeutically effective dose” of a mast cell desensitizing composition refers to an amount that is effective to achieve a desired therapeutic result. In some embodiments, a desired therapeutic result is a prevention or reduction of an inflammation in a subject. The reduction of inflammation can be a decrease by at least 10% as compared to a reference or control level, for example a decrease by at least about 20%, or at least about 30%, or at least about 40%, or at least about 50%, or at least about 60%, or at least about 70%, or at least about 80%, or at least about 90% or up to and including a 100% decrease. In some embodiments, the reference or control level is that obtained from an untreated subject or population.

In some embodiments, a desired therapeutic result is a prevention or reduction of a type I hypersensitivity reaction in a subject. The reduction of a type I hypersensitivity reaction can be a decrease by at least 10% as compared to a reference or control level, for example a decrease by at least about 20%, or at least about 30%, or at least about 40%, or at least about 50%, or at least about 60%, or at least about 70%, or at least about 80%, or at least about 90% or up to and including a 100% decrease. In some embodiments, the reference or control level is that obtained from an untreated subject or population. In some embodiments, a desired therapeutic result is systemic tolerance to the one or more type I hypersensitivity reaction antigens.

In some embodiments, a desired therapeutic result is a prevention or reduction of asthma, rhinitis, conjunctivitis, or dermatitis in a subject. The reduction of a asthma, rhinitis, conjunctivitis, or dermatitis can be a decrease by at least 10% as compared to a reference or control level, for example a decrease by at least about 20%, or at least about 30%, or at least about 40%, or at least about 50%, or at least about 60%, or at least about 70%, or at least about 80%, or at least about 90% or up to and including a 100% decrease. In some embodiments, the reference or control level is that obtained from an untreated subject or population.

In some embodiments, a desired therapeutic result is a prevention or reduction of anaphylaxis, urticaria, angioedema, food allergy, or drug allergy in a subject. The reduction of a anaphylaxis, urticaria, angioedema, food allergy, or drug allergy can be a decrease by at least 10% as compared to a reference or control level, for example a decrease by at least about 20%, or at least about 30%, or at least about 40%, or at least about 50%, or at least about 60%, or at least about 70%, or at least about 80%, or at least about 90% or up to and including a 100% decrease. In some embodiments, the reference or control level is that obtained from an untreated subject or population.

The therapeutically effective amount of the mast cell desensitizing composition and/or cytokine composition described herein can be determined by one of ordinary skill in the art and includes exemplary dosage amounts for a mammal of from about 0.5 to about 200 mg/kg of body weight of active composition per day, which can be administered in a single dose or in the form of individual divided doses, such as from 1 to 4 times per day. Alternatively, the dosage amount can be from about 0.5 to about 150 mg/kg of body weight of active composition per day, about 0.5 to 100 mg/kg of body weight of active compound per day, about 0.5 to about 75 mg/kg of body weight of active compound per day, about 0.5 to about 50 mg/kg of body weight of active composition per day, about 0.5 to about 25 mg/kg of body weight of active composition per day, about 1 to about 20 mg/kg of body weight of active composition per day, about 1 to about 10 mg/kg of body weight of active composition per day, about 20 mg/kg of body weight of active composition per day, about 10 mg/kg of body weight of active composition per day, or about 5 mg/kg of body weight of active composition per day.

In some embodiments, the mast cell desensitizing composition, the one or more type I hypersensitivity reaction antigens, and/or the one or more cytokines are administered intradermally or transdermally. In some embodiments, the mast cell desensitizing composition, the one or more type I hypersensitivity reaction antigens, and/or the one or more cytokines are administered intradermally or transdermally via microneedle or microneedle array. In some embodiments, the microneedle(s) is dissolvable. In some embodiments, the microneedle(s) used in the present invention is selected from those described in one or more of U.S. Pat. No. 8,834,423, PCT Publication No. WO 2017/120322, U.S. Publication No. 2018/0304062, U.S. Publication No. 2020/0353235, and PCT Publication No. WO 2021/178879.

It should be understood that more than one of the mast cell desensitizing composition, the one or more type I hypersensitivity reaction antigens, and the one or more cytokines can be administered concurrently or consecutively. Each of the mast cell desensitizing composition, the one or more type I hypersensitivity reaction antigens, and/or the one or more cytokines can be administered via different or the same route of administration. In a consecutive administration, the mast cell desensitizing composition, the one or more type I hypersensitivity reaction antigens, and/or the one or more cytokines can be administered in any order. In some embodiments, the method comprises a first administration of the one or more type I hypersensitivity reaction antigens and a second administration of a composition comprising the mast cell desensitizing composition. In some embodiments, the one or more type I hypersensitivity reaction antigens is administered first followed by the mast cell desensitizing composition and one or more cytokines. In some embodiments, the mast cell desensitizing composition, the one or more type I hypersensitivity reaction antigens, and/or the one or more cytokines are administered to the same cutaneous microenvironment of the subject.

All patents, patent applications, and publications referenced herein are incorporated by reference in their entirety for all purposes.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Methods

### Mice

Female C57BL 6, B6129P2-IL10^{tm1Cgn} (IL10 KO) (aged 6-8 weeks) mice were purchased from the Jackson Laboratories. C57BL 6-Mcpt5Cre⁺×Rosa26 DTA⁺ mice (provided by Axel Roers (Institute for Immunology, University of on Technology Dresden, Medical Faculty Carl-Gustav Carus, Dresden, Germany) were bred at the University of Pittsburgh. Mcp5tCre⁺ mice were crossed in-house with IL10fly mice (generously provided by Louise D'Cruz, University of Pittsburgh) to obtain Mcpt5Cre⁺×IL10^{fl/fl} mice.

C6(Cg)-IL10^{tm1.1.Karp/J} (VERT-X) mice (Jackson Laboratories) were bred at the University of Pittsburgh. On d0, mice were treated with vehicle on one ear and IgE on the contralateral ear. On day 1, mice were treated with either NKA (10 mg/50 mL) on both ears or vehicle on both ears. Three hours later, PCA was induced. On day 2, mice were euthanized. Excised Ear tissue was digested in IMDM media (Life Technologies) containing collagenase D (Sigma, 1.0 mg/mL) and DNAase (Roche, 1.0 mg/mL) at 37 C for 45 minutes. Single cell homogenates were blocked with FcBlock and stained. In some experiments, avidin AlexaFluro488 was injected intradermally to label cutaneous mast cells were labelled in vivo. Mice were euthanized 7 days after injection.

Age and gender matched mice were used for all in vivo studies. All experiments were in accord with the Guide for the Care and Use of Laboratory Animals and were approved by the Institutional Animal Care and Use Committee at the University of Pittsburgh.

### Culture and Activation of Murine MCs

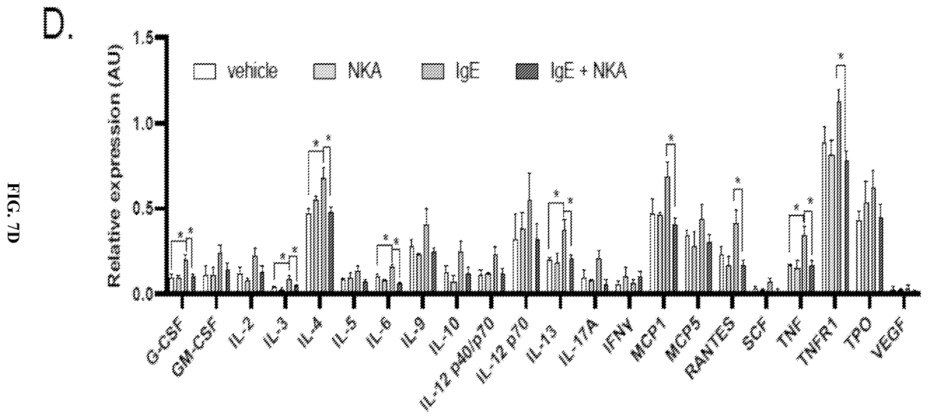

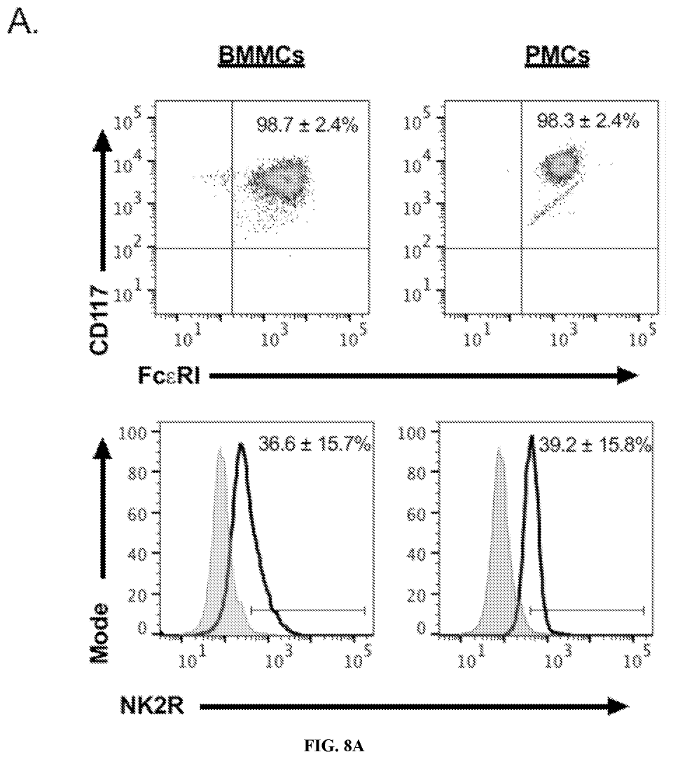

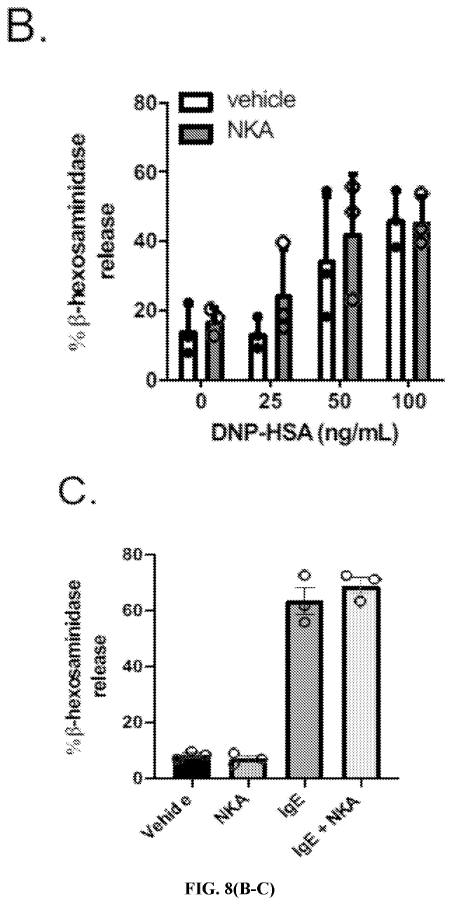

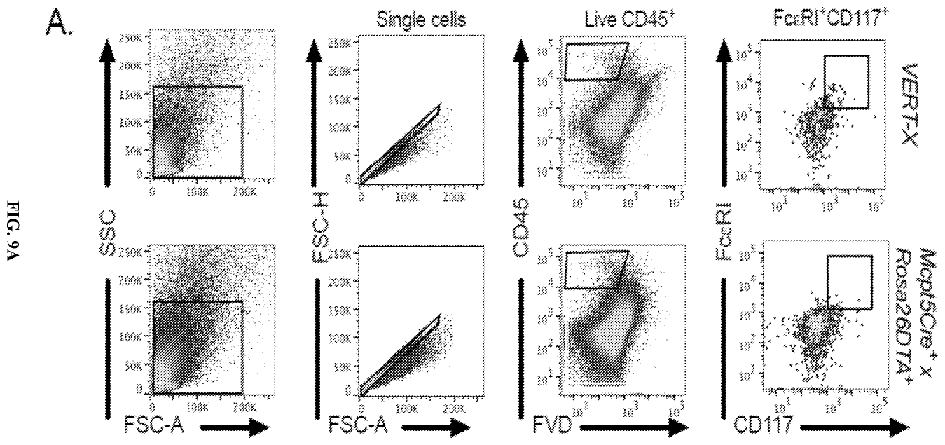

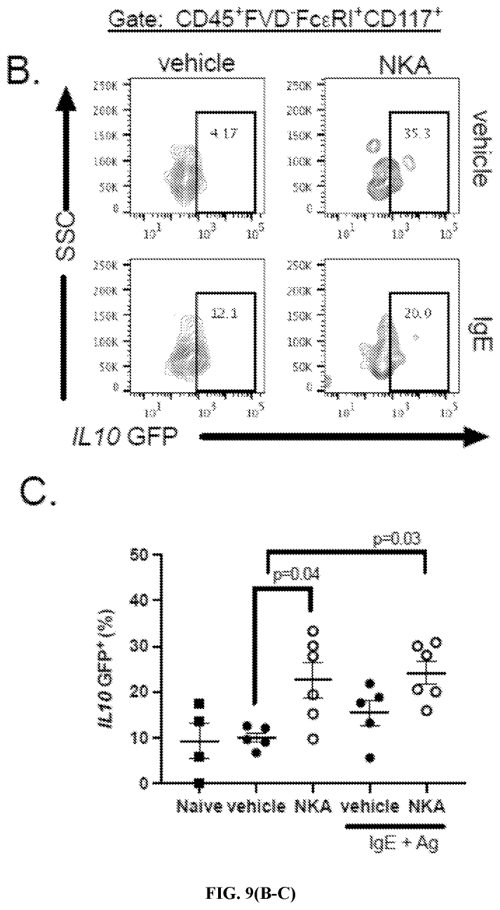

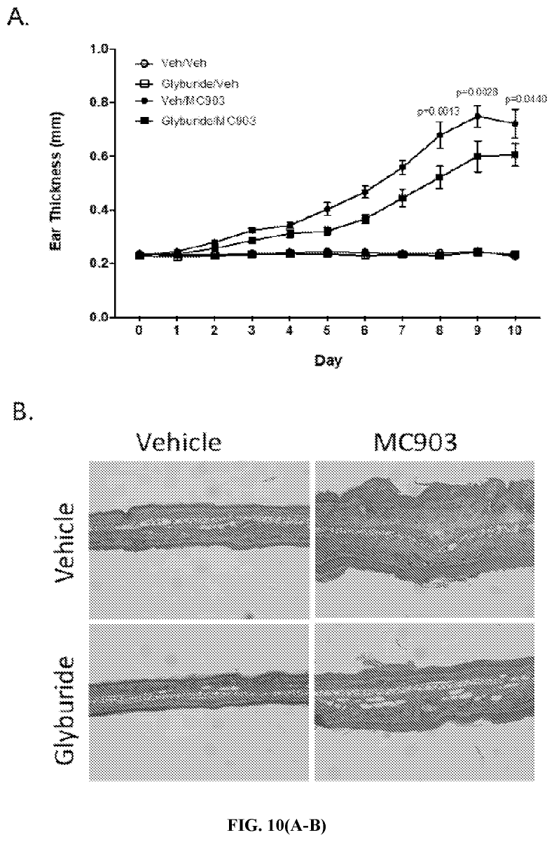

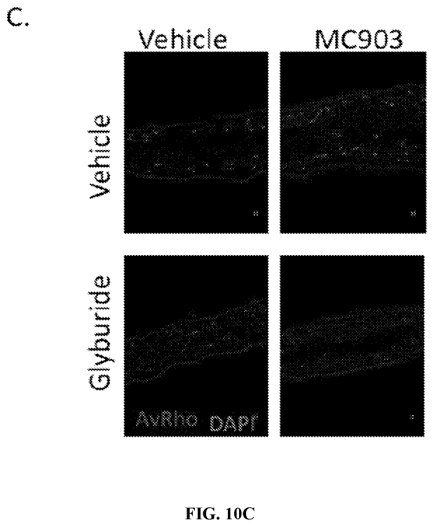

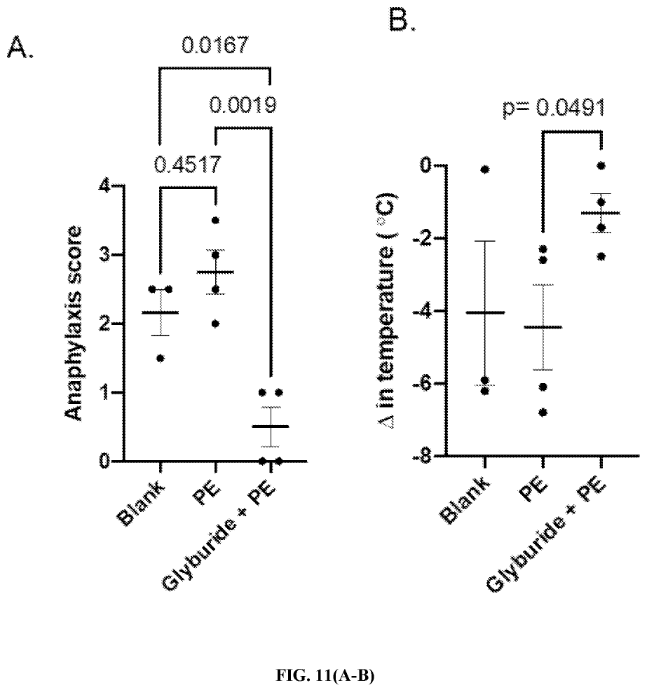

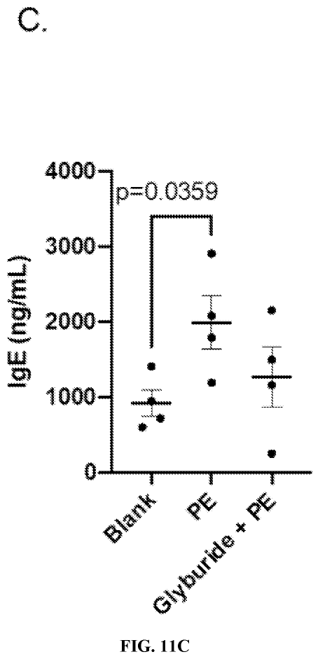

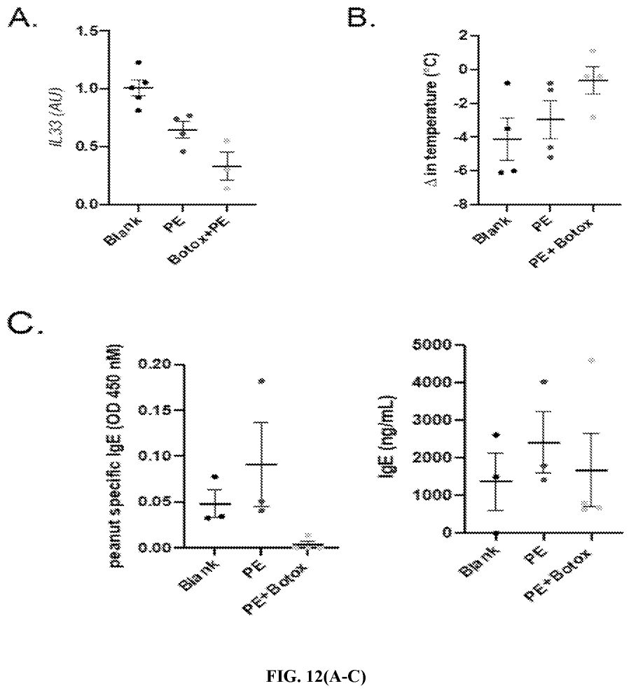

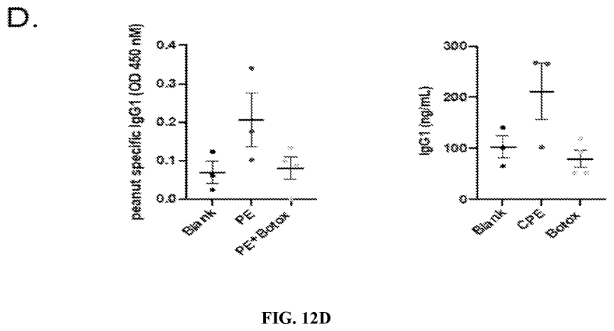

Mast cells were differentiated from murine bone marrow as has been described. In brief, marrow was expunged from the femurs and tibias of female mice. Cultures were maintained in complete MC/9 medium containing recombinant murine IL-3 (25 ng/mL, Peprotech) for 4-6 weeks prior to use. Peritoneal MCs (PMCs) were expanded from peritoneal washes with MC/9 medium supplemented with IL-3 (25 ng/mL) and SCF (Peprotech, 10 ng/mL) for 21 days. Purity of BMMCs and PMCs was confirmed to be greater than 95% (FIG. 8A).

To initiate signaling via the FcεRI, MCs were loaded with IgE (1.0 μg/ml, clone SPE-7, Sigma) for a minimum of 1 h then crosslinked with dinitrophenyl-human serum albumin (DNP-HSA, 100 ng/mL or as indicated). NKA (either LifeTein (Summerset, NJ) or Phoenix Pharmaceuticals (Burlingham, CA)) stock solutions were prepared in tissue culture grade water (100 μM) and stored at −20° C. for a maximum of 30 days. Glyburide (Cayman Chemical) was prepared in DMSO then diluted in PBS.

### Flow Cytometry and ImageStream

MCs were collected and washed with PBS containing 0.5% BSA and 0.1% sodium azide. Non-specific binding was inhibited using FcBlock (PBS with 2.0% goat serum and 0.25 mg/mL anti-CD16/32 (clone 2.4G2, BD Biosciences). Surface staining was done with anti-CD117 eFluor450 (clone 2B8, ThermoFisher), anti-FcεRI FITC (clone MAR-1, ThermoFisher) and anti-ABCA1 Alexa Fluor 647 (clone 5A1-1422, BioRad). Cells were washed with Wash Buffer (PBS with 0.5% BSA and 0.1% sodium azide). To detect differences in granulated proteins at the cell surface, IgE-loaded MCs were activated with DNP-HSA with or without NKA in Tyrode's Buffer for 30 min, washed with Wash Buffer then stained with Avidin Alexa Fluor488 (Invitrogen, Av488, 0.8 μg/5×10⁵ cells) and anti-LAMP-1 PE (clone eBio 1D48, ThermoFisher). Data were acquired on either a LSRII or a Fortessa (BD BioSciences). Analysis was performed with FlowJo v10.4 (BD Biosciences).

To detect phospho-Stat5Tyr694, MCs were loaded with IgE in serum and cytokine-free RPMI. Neurokinin A (1.0 μM) or vehicle control and DNP-HSA (100 ng/mL) were added simultaneously. At the indicated time, cells were fixed with 2.0% paraformaldehyde for 10 minutes then permeabilized with 90% methanol overnight at −20° C. Cells were stained with monoclonal anti-phospho-Stat5 Tyr694 Alexa Fluor 647 (clone C71E5, Cell Signaling Technology) for 60 minutes at room temperature. Nuclear co-localization was evaluated using ImageStream (Amnis). Activated BMMCs were fixed with 2.0% paraformaldehyde, permeabilized with Permeabilization Wash Buffer containing 3% FBS and 0.1% Triton X-100. and stained with anti-STAT5B Alexa Fluor 488 (clone EPR16671, Abcam) and DAPI. Data were analyzed using the Co-localization Wizard in IDEAS (v6.2). Co-localization for single cells expressing STAT5B and DAPI was quantified as Similarity Score, a log-transformed Pearson's correlation coefficient for the pixel intensity of corresponding images.

In other methods, mast cells differentiated for 5-7 weeks (BMMCs) or 3-4 weeks (PMCs) were collected, blocked with FcBlock and stained with anti-FceRI PE (clone MAR-1, Biolegend), anti-CD117 eFluor 450 clone 2B8, ThermoFisher) and anti-NK2R Alexa Fluor 647 or rabbit IgG Alexa Fluor 647 isotype control (Cell Signaling Technology). Single cell homogenates from the skin were stained with anti-CD45 BUV 395 (clone 30-F11, BD Biosciences), anti-FceRI Alexa Fluor 647 (clone MAR-1, Biolegend), anti-CD117 eFluor 450 and Fixable Viability Dye eFluor 780 (ThermoFisher) for 20 minutes at 4 C, washed, fixed with 2.0% paraformaldehyde then acquired on either a BD Fortessa or a LSR II cytometer.

### MS/MS on BMMC Releasate

BMMCs (4×10⁶) were activated in Tyrode's Buffer without BSA (500 μl) for 1 hour. Protease Inhibitor Cocktail (Sigma, containing aprotinin, bestatin, E-64, leupeptin and pepstatin A) was added to cell free supernatants. Supernatants were concentrated using Amicon Ultra Centrifugal Filters-3K (EID Millipore). Protein concentrations were quantified with the BCA Protein Assay Kit (ThermoFisher). Tryptic peptides were generated with the filter-aided sample preparation method, desalted with C18 spin columns and dried in aspeedvac. Peptides were reconstituted in 0.5% formic acid in 96:4 water:acetonitrile and resolved with liquid chromatography tandem mass spectrometry with a system composed of a Waters nanoACQUITY UPLC in-line with a Q-Exactive mass spectrometer (ThermoFisher). Solvent A (0.1% formic acid in water, Burdick & Jackson) and solvent B (0.1% formic acid in acetonitrile, Burdick & Jackson) were used as the mobile phase. Peptides were then eluted from a capillary column (100 μm inner diameter×100 mm long; ACQUITY UPLC M-Class Peptide BEH C18 Column, 1.7-μm particle size, 300 Å (Waters), and resolved using a 100-min gradient at a flow rate of 0.9 μL/min (4-33% B for 90 min, 33-80% B for 2 min, constant at 80% B for 6 min, and then 80-0% B for 2 min to equilibrate the column). Data were collected in positive ionization mode. PEAKSX software was used to sequence and identify peptides in each sample using a decoy search at a 1.0% false discovery rate using the UniProt murine database. Label-free quantitation was performed using the quantitative module in the PEAKSX software. The MS proteomics data have been deposited to the ProteomeXchange Consortium via the PRIDE partner repository with the dataset identifier PXD021377. Pathway analysis for expressed proteins was done using the Panther Classification System.

### Calpain Assay and ELISA

Calpain activity was detected from cell-free supernatants using the Calpain-Glo Protease Assay (Promega). Supernatants were incubated with substrate for 10 min prior to luminescent detection. In some experiments, glyburide or vehicle (0.1% DMSO) were added to cells 10 min prior to Ag. ELISA kits from eBioscience (IL-13), BioLegend, (TNF and IL-10) or RayBiotech (NKA) and used in accord with the manufacturer's recommendations. Data show pg/mL per 1×10⁶ cells.

### IL-10 Degradation Assays

To detect early IL-10 release, 4×10⁶ cells were incubated in serum free RPMI for a minimum of three hours prior to IgE loading and activation with DNP-HSA (100 ng/mL). Supernatants were collected and IL-10 was detected in accord with manufacturer's recommendations using 1.0% BSA to block and a top standard of 250 pg/mL. The lower limit of detection was 7.8 pg/mL. To evaluate the role of relationship between calpain and IL-10, cell-free supernatants were collected 10 min after activation and incubated with Ac-calpastatin (Ac-Cal; 20 μg/mL, Cayman) or vehicle control. Ac-Cal containing supernatants were further incubated for 30 min prior to IL-10 quantification by ELISA. To directly test IL-10 proteolysis, MCs were activated for 45 min, cell free supernatants were collected and pulsed with recombinant IL-10 (Biolegend, 250 pg/mL).

The IL-10 degrading capacity of calpain, recombinant murine IL-10 (Peprotech) was incubated with human calpain I (Athens Research and Technology) for 60 min at 37° C., then denatured with Laemelli Buffer (BioRad) and resolved on 10% Tris-Glycine SDS-PAGE gels (BioRad). Proteins were transferred to 40 μm PVDF membranes (BioRad), blocked for 1 h with Odyssey Blocking Buffer (LI-COR) and probed overnight with goat anti-mouse IL-10 antibody (RnD Systems). Ant-IL10 was detected with donkey anti-goat 800CW (LI-COR) and visualized on a LI-COR Imaging System. Densitometry was calculated using ImageStudioLite v5.2.5 (Licor). Values are presented as (rIL-10+calpain)/rIL-10 alone)*100.

### RT-PCR

Total RNA was isolated from either MCs or murine ear tissue 3 h hours after NKA injection. Tissue was homogenized in Navy RINO tubes (Next Advance) in a Bullet Blender Storm (Next Advance). RNA was extracted per standard methods with Trizol (Invitrogen). cDNA was reversed transcribed with the Quantitech Reverse Transcription Kit (Qiagen). PCR products were amplified with Fast SybrGreen (ABI) on a StepOne Plus Cycler (BioRad) using the following primers: TNF F: CCGATGGGTTGTACCTTGTC (SEQ ID NO: 5), TNF R: CGGACTCCGCAAAGTCTAAG (SEQ ID NO: 6); IL13 F: CCTGGCTCTTGCTTGCCTT (SEQ ID NO: 7), IL13 R: GGTCTTGTGTGATGTTGCTCA (SEQ ID NO: 8); IL10 F: GCTGGACAACATACTGCTAACC (SEQ ID NO: 9); IL10 R: ATTTCCGATAAGCTTGGCAA (SEQ ID NO: 10); Bactin F: GGCTGTATTCCCCTCCATCG (SEQ ID NO: 11); Bactin R: CCAGTTGGTAACAATGCCATGT (SEQ ID NO: 12). Data are presented as 2^{-ΔΔCT} using f-actin as the housekeeping gene and normalizing to either untreated cells or naïve control mice.

### Passive Cutaneous Anaphylaxis

Mouse ears were measured with a micrometer then sensitized with IgE in filter-sterilized PBS (Sigma, clone SPE-7, 20 ng/50 μl/ear) or vehicle control. Twenty-four hours later mice were treated with NKA (10 μg/ear, intradermal) or vehicle (PBS) 3 h prior to Ag (DNP-HSA 100 μg, in 200 μl, intravenous in saline) administration. Data are presented as change in ear thickness between baseline and the indicated time. Where indicated, mice were treated with recombinant murine IL-10 (Peprotech, 1.0 ng/ear) at the time of IgE injection or treated with glyburide (2.5 mg/kg, i.p), 3 h prior to Ag administration.

Edema was evaluated histologically and as a function of Evans blue dye extravasation. For histological evaluation, mice were euthanized 2 h after PCA induction. Paraformaldehyde (4.0%) fixed ears were processed for hematoxylin and eosin staining. Pathology was blindly evaluated on an Axiostar plus microscope equipped with epifluorescence and a digital camera (AxioCam; Zeiss). For dye extravasation, Ag was delivered in 1.0% Evans blue dye. Ninety min later mice were euthanized, and ears were excised. Dye was extracted in DriSolv (EMD Millipore) and quantified as absorbance at OD 650 nm. Data are shown as OD650/mg of tissue.

Cytokine expression in the skin was investigated 24 h after PCA induction. Mice were euthanized, ears excised, and tissue homogenized in T-PER (ThermoFisher) with Protease Inhibitor Cocktail (Sigma). Protein concentrations were quantified using the BCA Protein Assay (Pierce). The protein profile of skin homogenates was characterized with C-Series Mouse Cytokine Antibody Array C1 (RayBiotech). Densitometry values subtracting the median background from individual spot areas were calculated with ImageStudioLite v5.2.5. The densitometry value from each cytokine spot was divided by the mean densitometry of positive control spots to give relative protein expression. Data were from each experiment were normalized as (Relative Expression [Treated]/Relative expression [Vehicle]). In some experiments, ELISAs were performed on skin homogenates.

### Myeloperoxidase Activity Assays

Neutrophil influx was evaluated as a function of myeloperoxidase (MPO) activity. To detect MPO activity, tissue was homogenized with a Next Advance Bullet Homogenizer in 50 mM KPG₄ with 50 mM hexadeyltrimethylammonium. Homogenate was incubated with ODP (Sigma) with 0.005% H₂O₂ for 30 minutes. Enzymatic reactions were stopped with 1N HCl and absorbance was read at 450 nM. Data show MPO activity/mg of tissue.

b-Hexosaminidase Assays

Mast cells (5×10⁵-1×10⁶) were loaded with IgE (1.0 mg/mL) for a minimum of 3 h in Tyrode's Buffer. Neurokinin A (1.0 mM) was added with DNP-HSA (Sigma, as indicated or 100 ng/mL) for 40 min at 37 C. Cells and supernatants were collected, cell pellets were lysed in Tyrode's Buffer by freeze-thawing then incubated with p-nitrophenyl N-acetyl j-D-glucosaminide (EMB Millipore, 5 mM) dissolved in citrate buffer (0.04 M, pH 4.5) for 90 minutes. The enzymatic reaction was stopped with glycine (0.4 M, pH 10.7). Absorbance at 450 nM was read. The average percentage of b-hexosaminidase release from triplicate values was calculated as (ODₛᵤₚ/ODₛᵤₚ+OD_{lysate})×100(%).

### Statistics

Data were analyzed by either t-test or by 2-way ANOVA with Boneforroni post-hoc analysis using Prism GraphPad (v 8.0).

### Example 2. Neurokinin a Increases in the Skin Following Mast Cell Activation

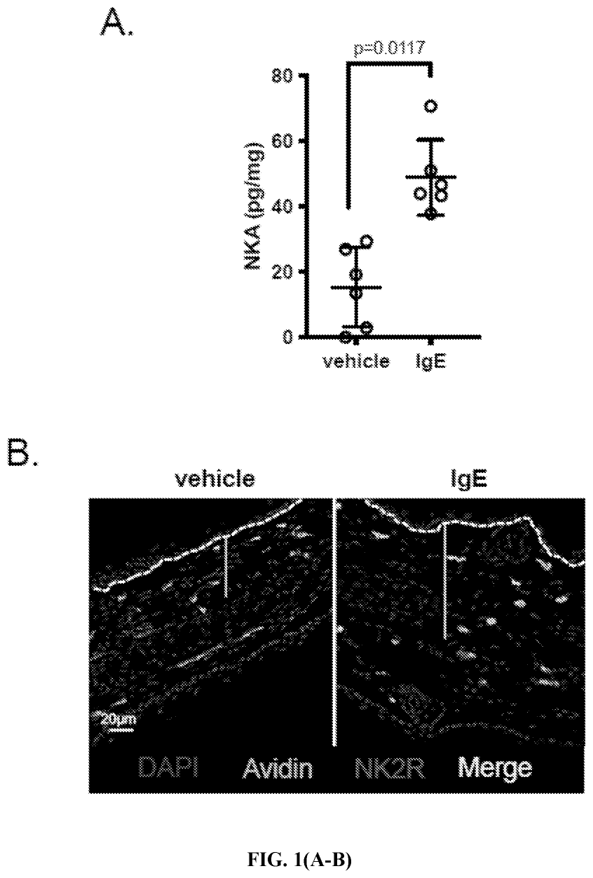

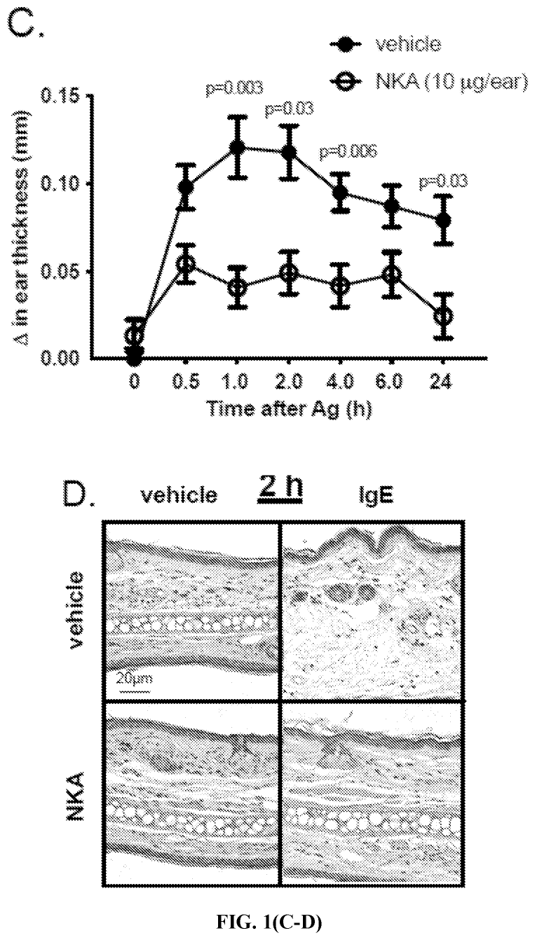

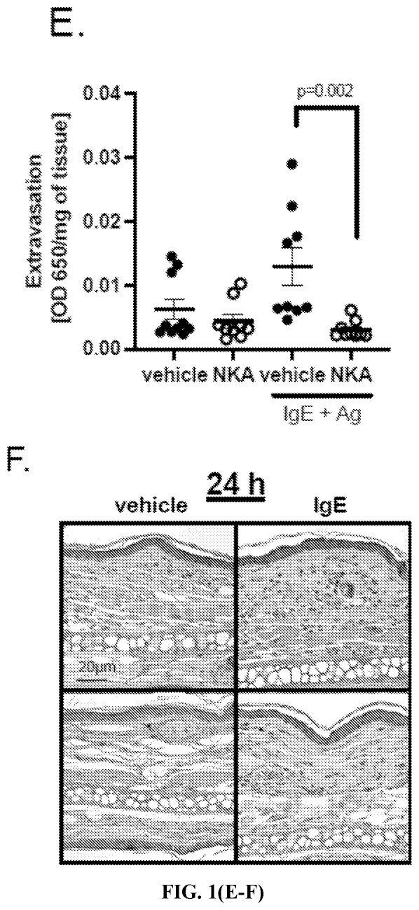

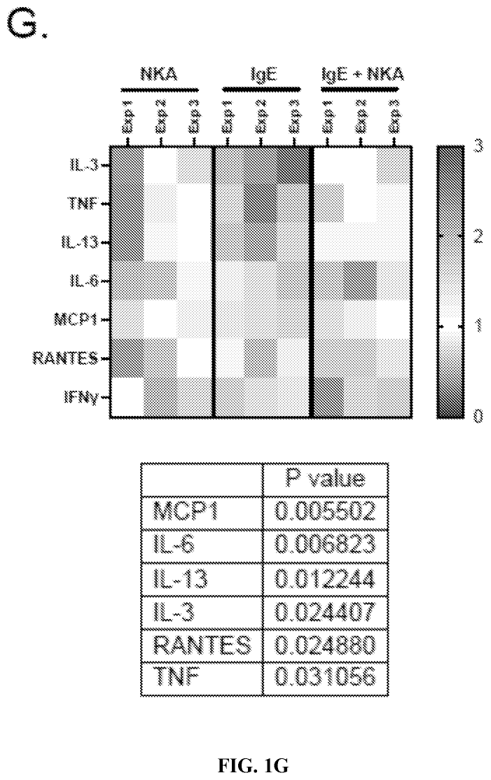

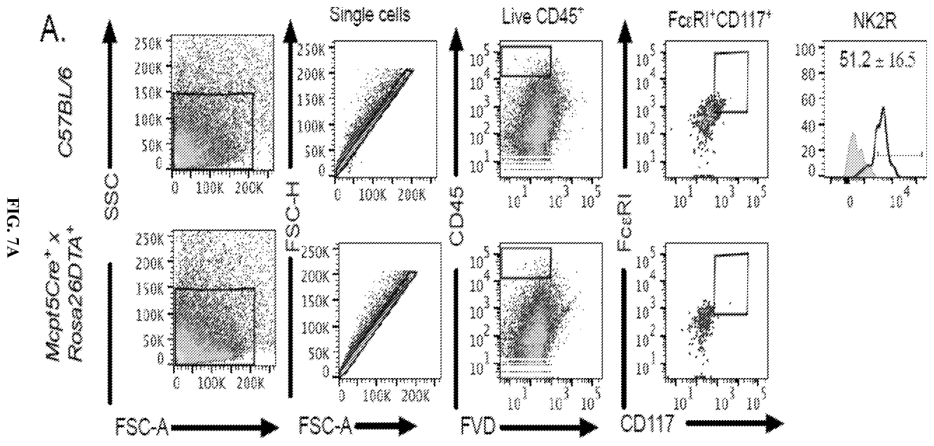

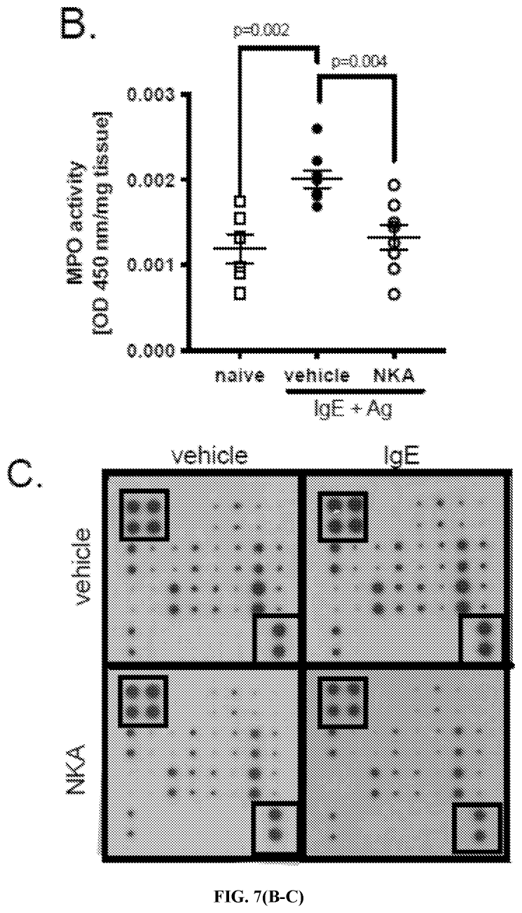

Neurokinin A is released by peripheral sensory neurons and has been detected in human and rat skin, though its expression has not been reported in murine skin. To confirm expression in murine skin and evaluate a potential relationship between MC activation and NKA expression, the level of NKA in mouse skin homogenates was investigated in the steady state and following IgE-initiated mast cell activation with the passive cutaneous anaphylaxis model (PCA). Basal levels of NKA were near undetectable. But, 24 hours following MC activation, NKA levels increased (FIG. 1A). NK2R expression on MCs was confirmed by immunoflurescent microscopy (FIG. 1B) and by flow cytometry (FIG. 7A). These data suggest that inflammation induced by MC activation regulates local NKA levels and that MCs are equipped to respond to changes in NKA in the microenvironment.

### Example 3. Exogenous Neurokinin a Downregulates MC-Function in PCA

Neurokinin A inhibits inflammation in a model of allergic CHS which is characterized by MC infiltration and activation. It is demonstrated herein that NKA is upregulated in PCA and it was hypothesized that NKA may naturally inhibit MCs activation. To test this hypothesis, mice were pretreated with NKA three hours prior to PCA induction with concentrations that inhibit CHS. Neurokinin A pretreatment inhibited the early (1-2 hours) phase of PCA seen as reduced ear thickness, a reduction in edema in histological sections and reduced Evans blue dye extravasation (FIG. 1C-E). Neurokinin A pretreatment also diminished the late phase of PCA (24 h) reflected in reduced ear thickness, reduced neutrophil influx seen in histological sections and reduced myeloperoxidase activity (FIG. 1B, IF, FIG. 7B). The effects of NKA pretreatment on late phase PCA were further characterized using a protein array to evaluate inflammatory cytokines. When IgE-treated skin was compared with vehicle treated skin 24 h after PCA induction, cytokines associated with MC-survival (IL-3), Th2 immunity (IL-13, IL-4), chemotaxis (MCP1 and RANTES) and innate inflammation (TNF, G-CSF, IL-6) increased significantly (FIG. 1G, FIG. 7C, D). Neurokinin A pretreatment significantly reduced IL-3, IL-6, IL-13, MCP1, RANTES and TNF in IgE-treated skin. Though not statistically significant, IFNγ levels increased in NKA-treated skin, consistent with reports demonstrating IFNγ induction by NKA in the lung.

### Example 4. Neurokinin a Inhibits STAT5 Activation and Subsequent Transcription in BMMCs

In PCA, NKA inhibited the release of IL-3, thought to be produced by T cells, as well as a panel of MC-produced cytokines: IL-6, IL-13, TNF, RANTES, MCP1. In MCs and in other cells, the production of these cytokines and chemokines is associated with a STAT5 activation. Therefore, it was hypothesized that NKA may inhibit the phosphorylation of STAT5 and downstream transcription of factors associated with allergy and inflammation. To test this hypothesis, an in vitro approach was utilized to directly evaluate the effects of NKA on FcεRI-activated BMMCs to test this hypothesis. IgE-activated BMMCs which are readily expanded in vitro were utilized and confirmed key findings with connective tissue type PMCs which better reflect MCs in the skin but expand less robustly. Expression of the NK2R was equivalent on both BMMCs and PMCs (FIG. 8A).

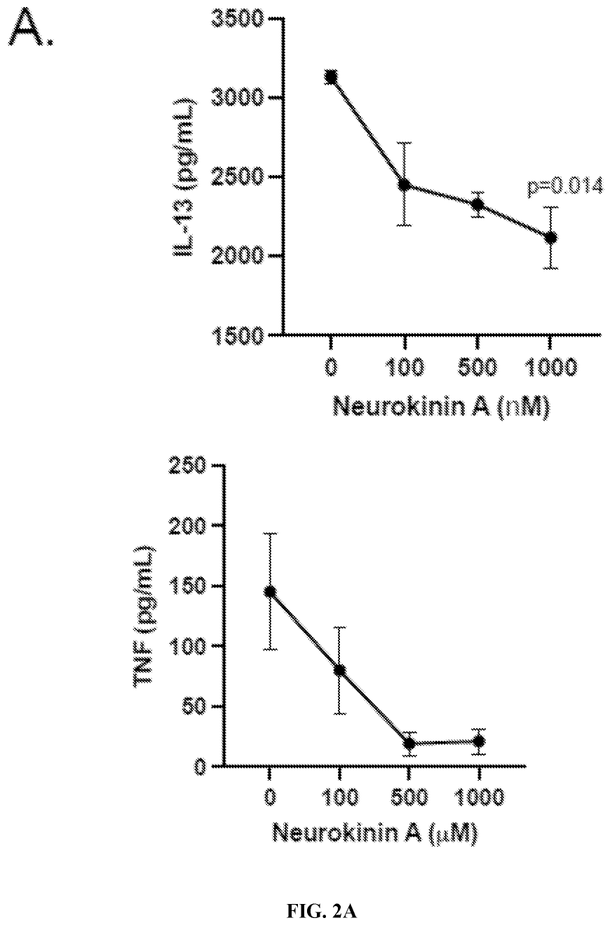

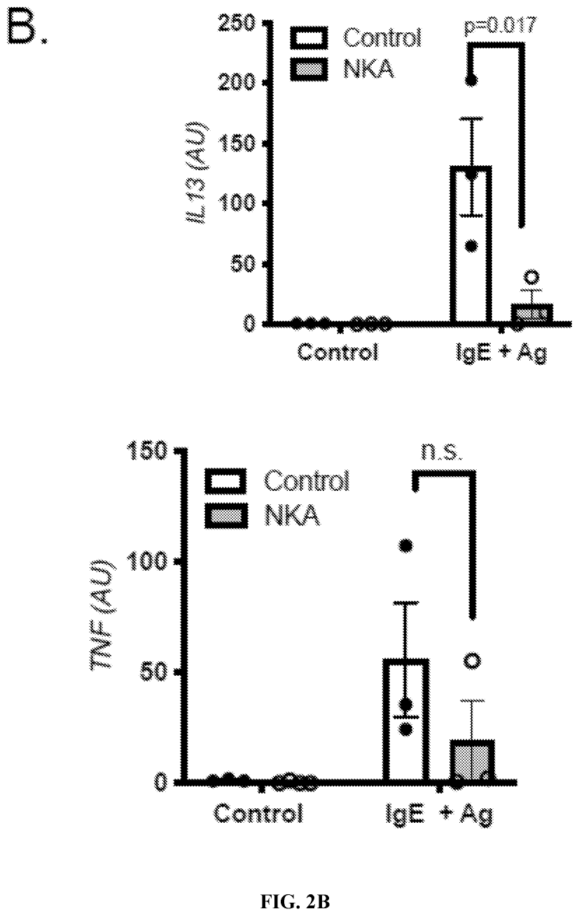

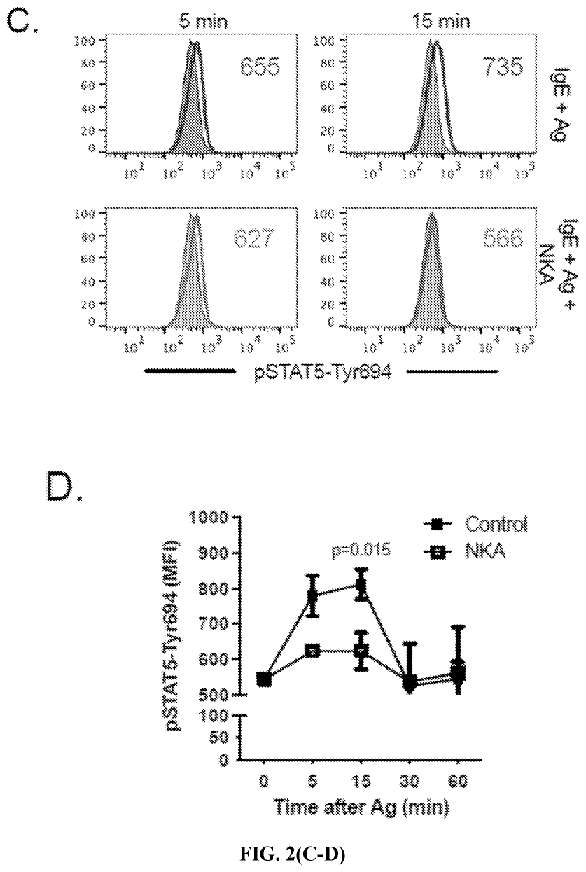

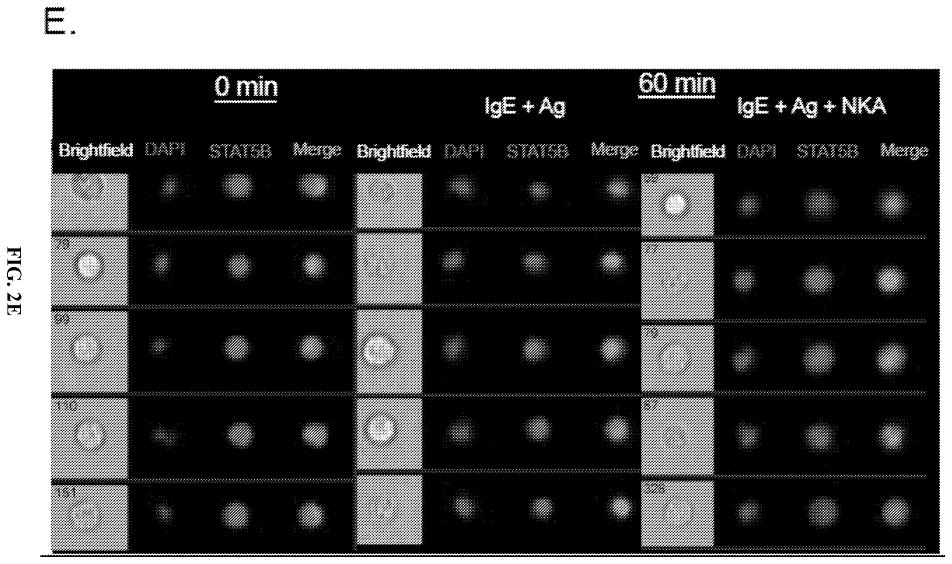

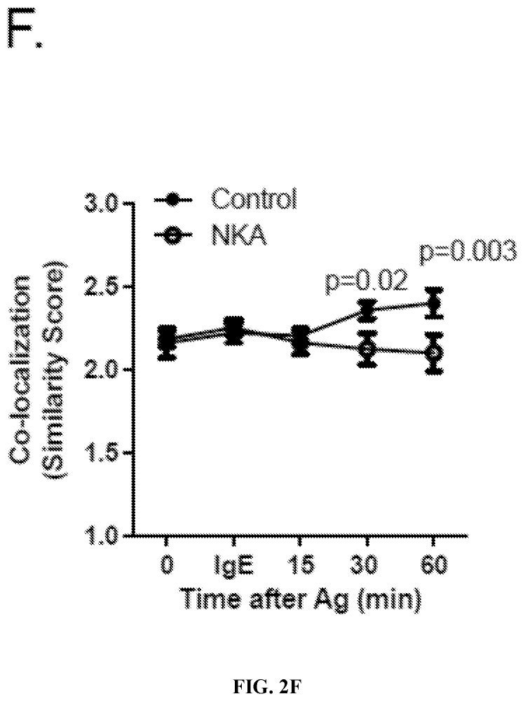

First, the in vivo finding that NKA inhibited the release of MC-derived cytokines was confirmed. TNF and IL-13 levels were focused upon, representing the innate and allergic inflammatory functions of MCs. After overnight activation with IgE+Ag in the presence of NKA and transcription 60 min after activation. IL-13 and TNF release (FIG. 2A) and transcription (FIG. 2B) were both inhibited by NKA. Phosphorylation and nuclear translocation of STAT5 are upstream of IL13 and TNF transcription In FcεRI-activated MCs. Accordingly, 15 minutes after crosslinking IgE with Ag, pTyr695-STAT5, detected by flow cytometry peaked in control BMMCs and was reduced when MCs were activated in the presence of NKA (FIG. 2C-D). The nuclear translocation of STAT5B, the dominant STAT5 in MCs was evaluated with imaging flow cytometry. STAT5B increased 30 and 60 minutes following IgE crosslinking and was significantly reduced in BMMCs activated in the presence of NKA (FIG. 2E-F). Collectively, these data suggest that NKA inhibits STAT5 activation, transcription and release of the pro-inflammatory and Th2 skewing cytokines, IL-13 and TNF.

### Example 5. NKA Inhibits MC Activation Through an IL-10-Dependent Mechanism

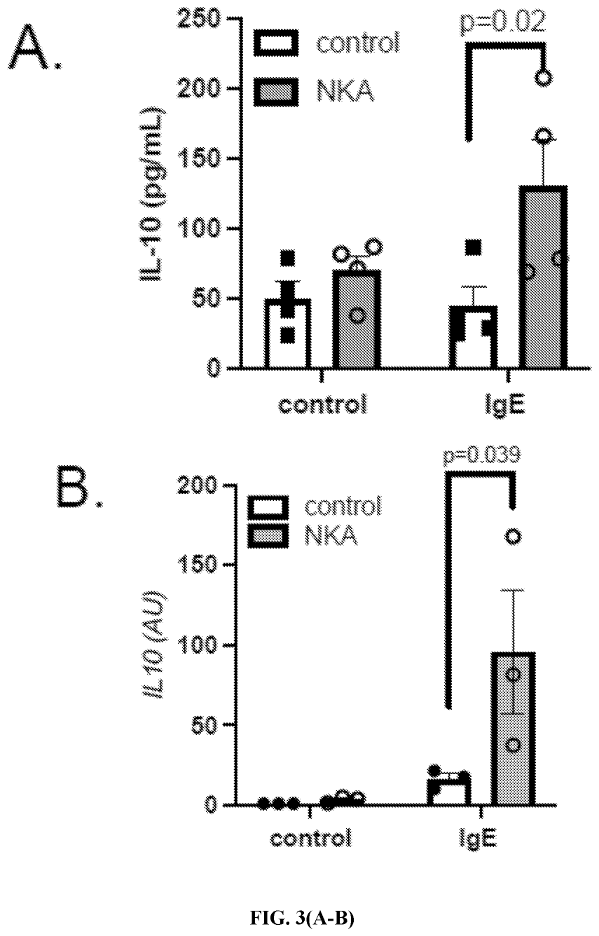

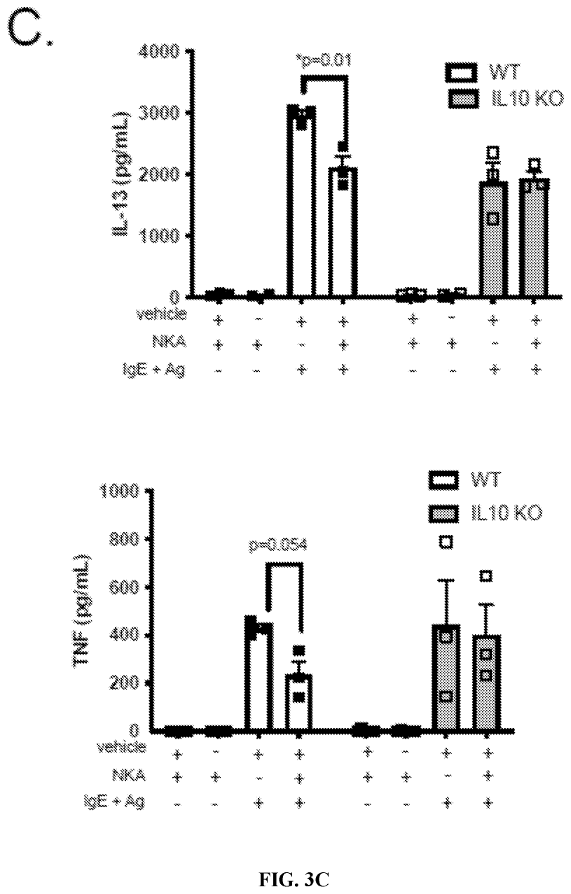

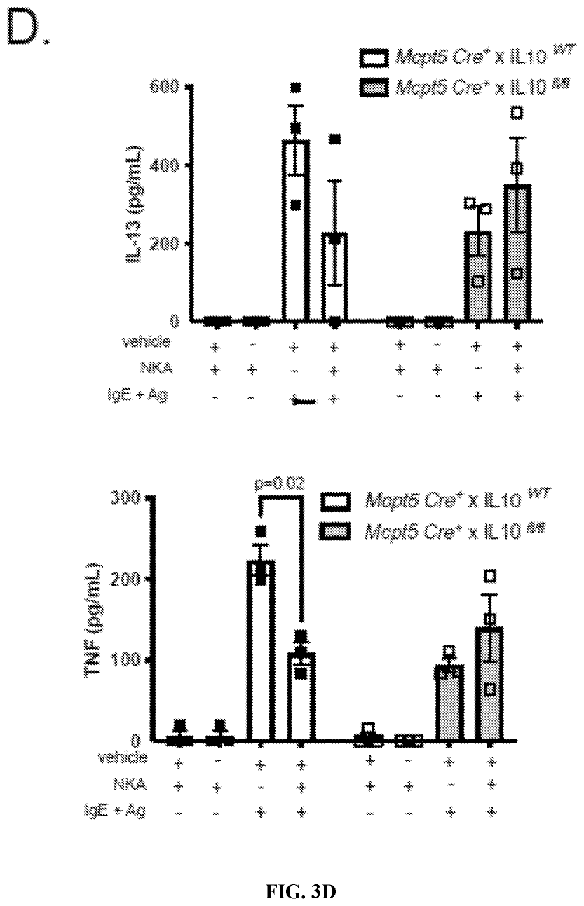

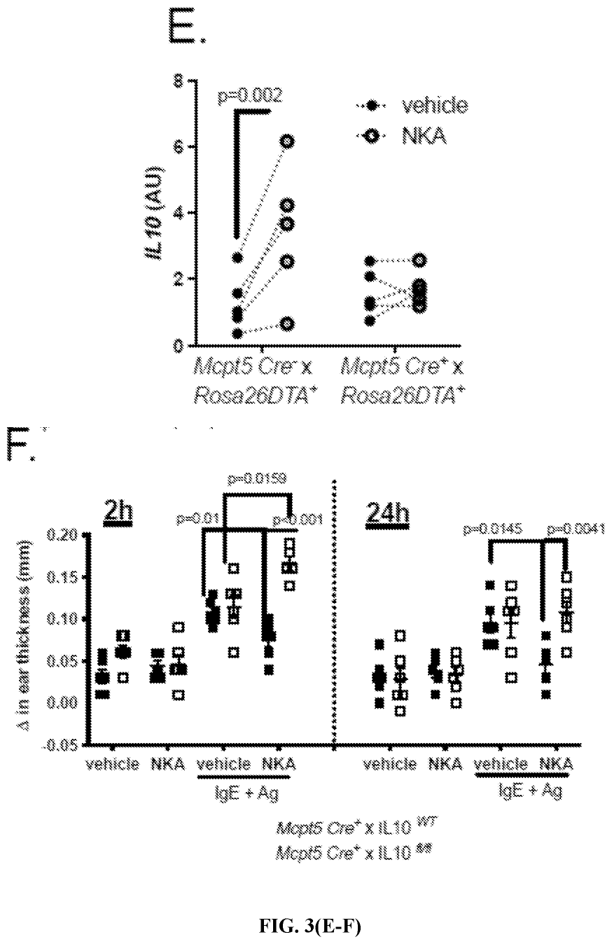

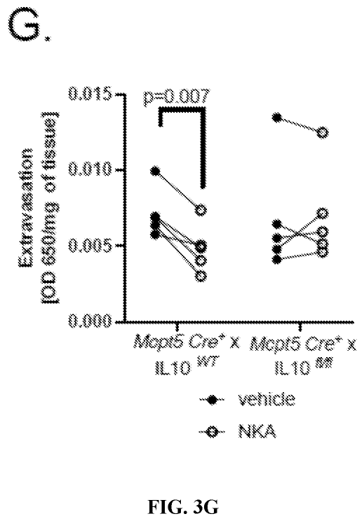

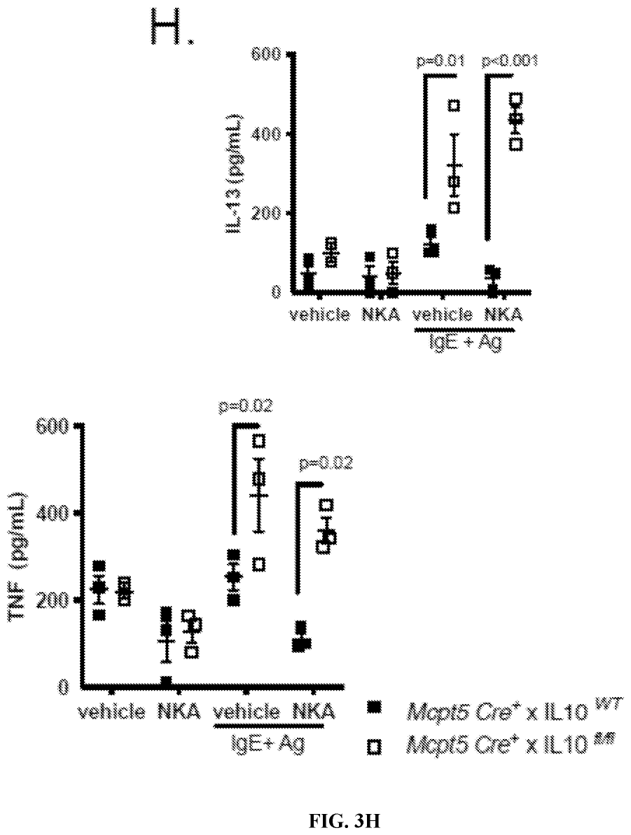

Immunoregulatory cytokines such as IL-10 inhibit FcεRI-initiated STAT5 phosphorylation. Evaluation of IL-10 in supernatants from BMMCs activated following overnight culture with NKA showed a significant increase in IL-10 protein and increased IL10 RNA 60 min after activation (FIG. 3A-B). The functional role for IL-10 in NKA-mediated suppression was addressed with IL-10 KO BMMCs. In response to IgE-initiated activation, IL-10 KO BMMCs released significantly lower levels of IL-13 and TNF compared to WT BMMCs (FIG. 3C), consistent with previous reports. In absence of autocrine IL-10, NKA did not inhibit IL-13 or TNF release (FIG. 3D). This observation was confirmed using PMCs expanded from Mcpt5Cre⁺×IL10^{WT} and Mcpt5Cre⁺×IL10^{fl/fl} mice, in which a MC-specific Cre drives IL-10 deletion. As observed for BMMCs, NKA inhibited IL-13 and TNF release from control Mcpt5Cre⁺×IL10^{WT} PMCs (FIG. 3E). Without PMC-derived IL-10, the effects of NKA were reversed. Collectively, these data demonstrate that autocrine IL-10 is required for NKA to inhibit FcεRI-initiated MC activation.

### Example 6. MC-derived IL-10 is required for NKA-initiated inhibition of PCA

The relationship between NKA and IL-10 in PCA was then investigated. In contrast to the in vitro observations on MCs, in total skin homogenates, IL-10 protein did not increase in NKA pretreated mice (FIG. 7E). However, with this broad screen the contributions of autocrine, MC-derived IL-10 may have been masked by other cells in the skin. Alternatively, that MC-derived IL-10 may be important at earlier timepoints. To more specifically evaluate the capacity for NKA to regulate IL-10 from MCs, IL10-eGFP reporter mice were used. Though this line is a transcriptional reporter, the expression of eGFP correlates with protein. The percentage of IL10-eGFP detected by this method was low in MCs from naïve mice and increased following PCA induction. Neurokinin A specifically increased the percentage of IL10-eGFP⁺ MCs two-fold with and without PCA induction (FIG. 9A-C). To corroborate this finding, IL10 mRNA was evaluated in the skin of MC-sufficient (Mcpt5Cre⁻×Rosa26DTA⁺) and MC-deficient (Mcpt5Cre⁺×Rosa26DTA⁺) mice treated with vehicle or NKA (FIG. 3E). While NKA significantly increased IL10 mRNA levels in MC-sufficient mice, there was no difference in IL10 mRNA in MC-deficient mice, suggesting that NKA increases IL-10 in the skin in a MC-dependent manner.

The function of autocrine IL-10 in NKA-mediated PCA inhibition was evaluated using Mcpt5Cre⁺×IL10^{WT} and Mcpt5Cre⁺×IL10^{fl/fl} mice. Mcpt5Cre⁺×IL10^{WT} and Mcpt5Cre⁺×IL10^{fl/fl} mice have comparable numbers of CD45+cells and MCs (not shown). The immune-regulatory capacity of NKA was lost at 2 h and 24 h after PCA induction (FIG. 3F). While NKA inhibited Evans blue dye extravasation in Mcpt5Cre⁺×IL10^{WT} mice, it had no effect on extravasation in Mcpt5Cre⁺×IL10/l mice (FIG. 3G). By cytokine array, IL-13 and TNF were upregulated during PCA and down-regulated by NKA. ELISA was used to confirm that these proteins were significantly downregulated by NKA in Mcpt5 Cre⁺×IL10^{WT} mice (FIG. 3H). In the absence of MC-derived IL-10, the administration of NKA increased IL-13 and TNF in the skin. Collectively, these data suggest that MC-derived IL-10 is required for the inhibitory effects of NKA.

### Example 7. Endogenous NKA is Regulated by IL-10 in the Skin

The data herein show that NKA inhibits MC-activation in an IL-10 dependent manner. Little is known about the regulation of NKA in the skin. Therefore, it was next asked if there was a relationship between IL-10 and NKA in vivo. To address this, NKA levels were evaluated by ELISA in the steady state and after PCA induction in WT and IL-10 KO mice. In skin homogenates from WT mice, NKA levels are low in the steady state and increase significantly following PCA (FIG. 1B, FIG. 31). In skin homogenates from IL-10 KO mice, NKA remains low after PCA induction. Conversely, administration of exogenous recombinant IL-10 significantly increased steady state NKA without PCA induction (FIG. 31). Collectively, the data illustrate a cyclic relationship between IL-10 and NKA.

### Example 8. NKA has Diverging Effects on Granule Release in BMMCs and in PMCs

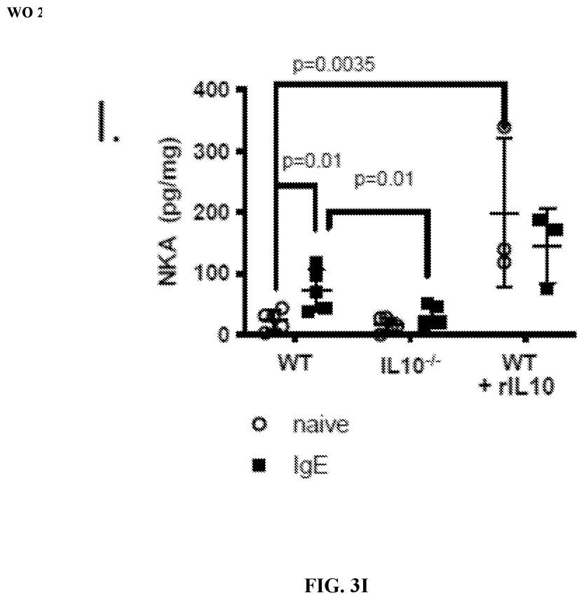

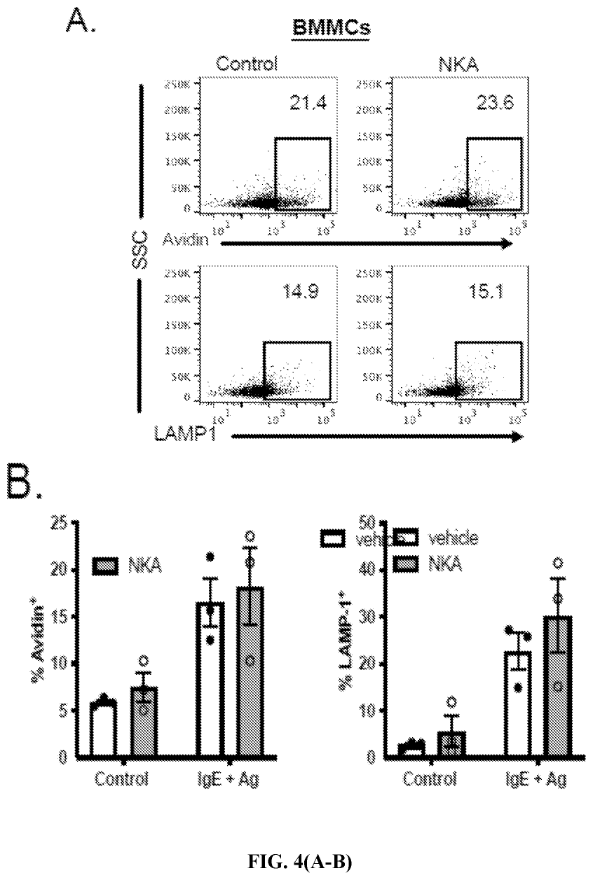

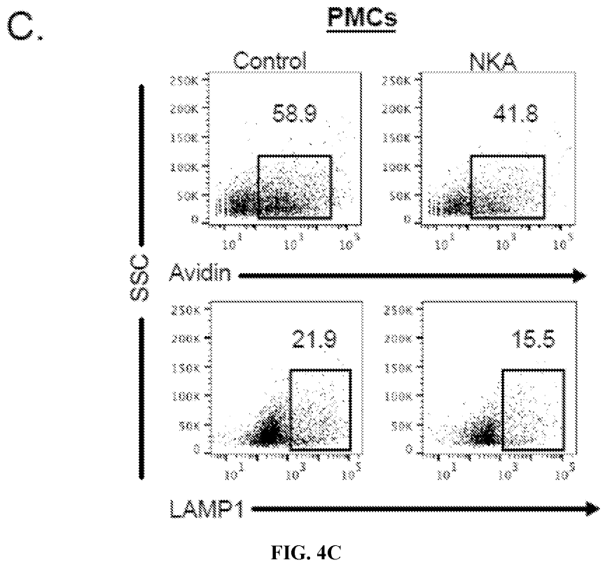

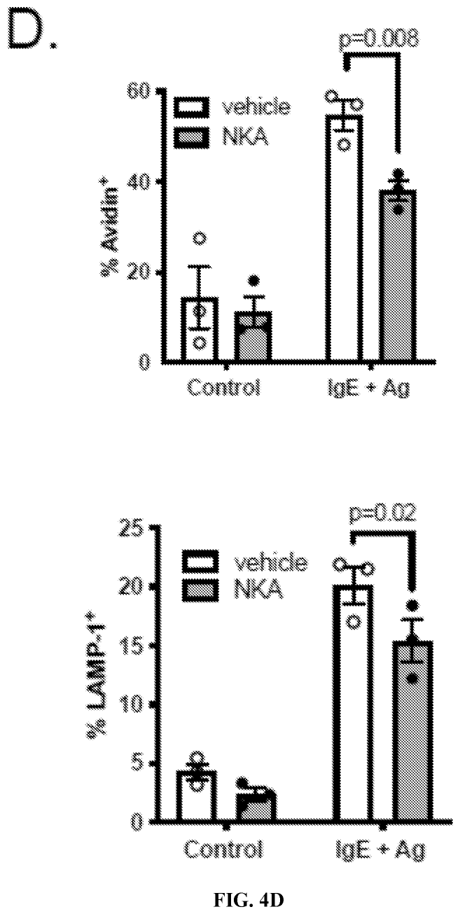

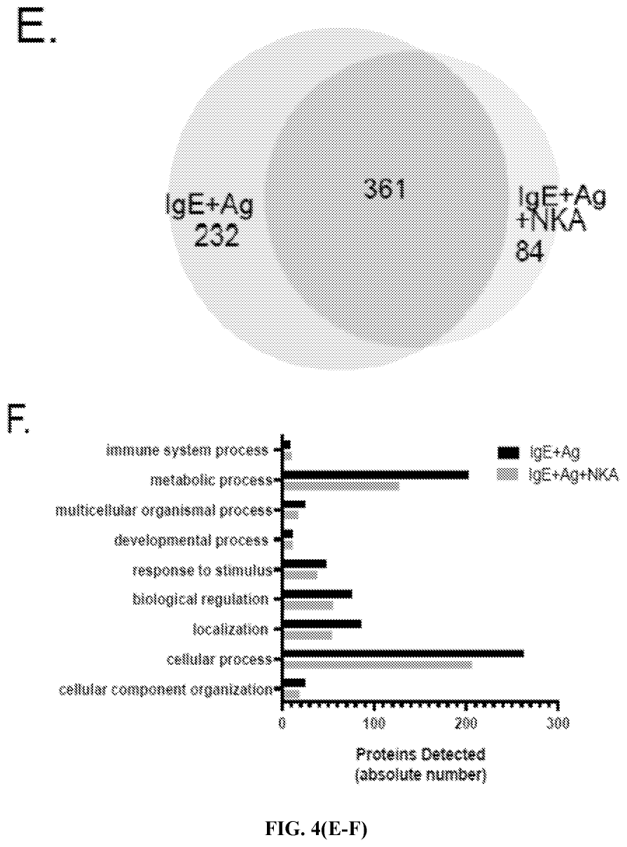

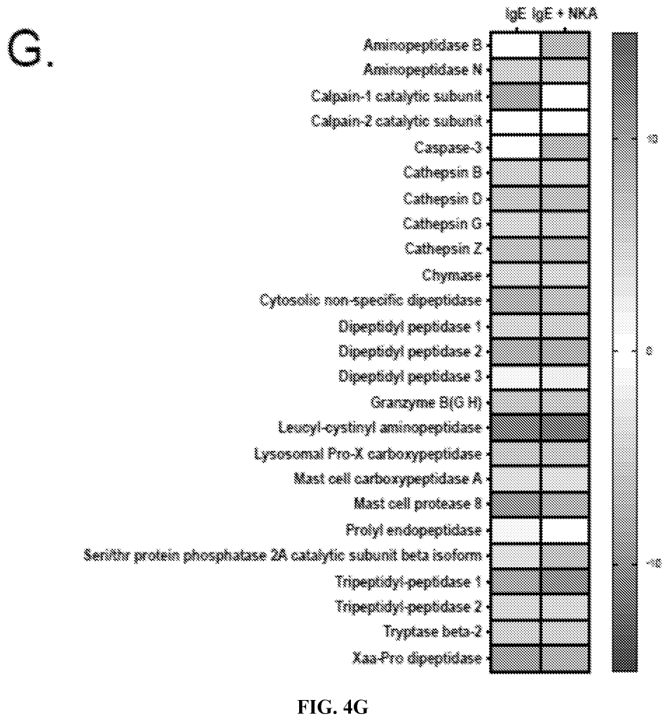

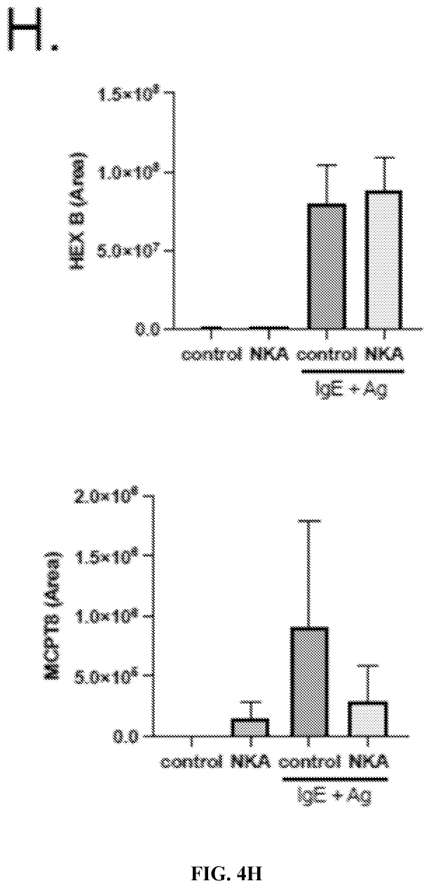

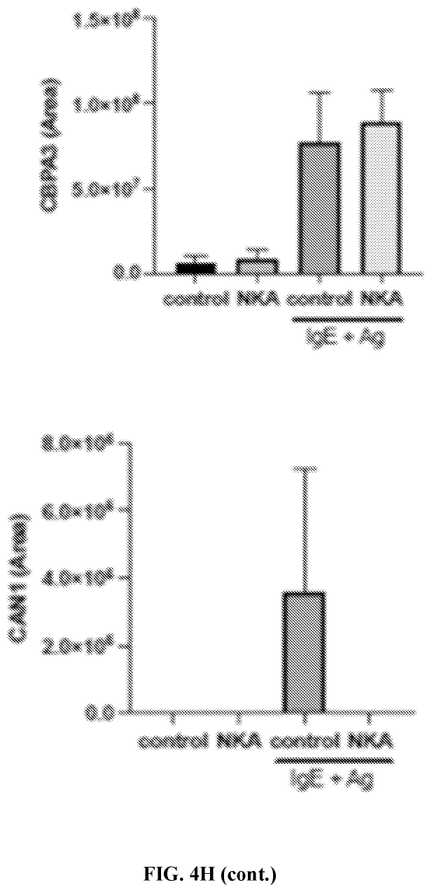

Neurokinin A fully inhibited PCA, including degranulation-associated edema. The effects of NKA on IgE-initiated degranulation in vitro was next examined. Classic studies suggest that NKA alone does not affect intracellular calcium flux, β-hexosaminidase or histamine release in MCs. In line with this observation, we found no difference in β-hexosaminidase release from BMMCs or PMCs incubated with NKA or activated with IgE and antigen in the presence of NKA (FIGS. 8B and 8C). It was hypothesized that NKA affects other pathways of degranulation. To test this hypothesis, granulated proteins budding from the cell surface 30 min after activation were labelled with avidin, and exocytotic vesicles fusing with the cell membrane were labelled with anti-LAMP1. BMMCs activated with or without NKA expressed equivalent amounts of avidin⁺ granules and LAMP-1 on the cell surface (FIG. 4A-B). In contrast, NKA reduced the percentage of avidin⁺ and LAMP-1V PMCs compared to control (FIG. 4C-D).

### Example 9. Shotgun Proteomics Identify Pathways Affected by NKA During MC Activation

Neurokinin A inhibited cytokine release in an IL-10-dependent manner from both BMMCs and PMCs but had diverging effects on exocytosis. It was hypothesized that NKA affected a shared pathway early during activation that was independent of conventional hallmarks of degranulation. To identify NKA-regulated proteins, shotgun proteomics was performed to better understand the role of NKA on mediator release initiated by FcεRI-activation. BMMCs were chosen for this screen because they expand in greater numbers than PMCs, more readily providing the amounts of protein necessary for this type of screen. With this broad screen 232 proteins uniquely released in IgE activated supernatants and 94 proteins were upregulated by NKA (FIG. 4E, top 20 proteins released in each condition summarized in Tables 1-2). Pathway analysis of proteins detected following activation suggested that NKA inhibited the release of proteins associated with two pathways: metabolism, including mitochondrial proteins and cellular function, which includes proteases, conventionally released by IgE-activated MCs (FIG. 4F). The relationship between neuropeptides and the release of mitochondrial proteins has previously been reported. Therefore, the analysis focused on the later classification of proteins performing semi-quantitative analysis for proteases, normalizing the coverage area of proteins identified from activated supernatants to that from controls (FIG. 4G). The release of proteases commonly associated with BMMC activation including β-hexosaminidase, MC carboxypeptidase, cathepsin D and chymase were unchanged by NKA (FIG. 4H). However, the catalytic subunit of the cysteine protease, calpain 1 was detected at varying levels from BMMCs activated with IgE+Ag but not detected from BMMCs activated in the presence of NKA.

### Example 10. Extracellular Calpain is Regulated by NKA

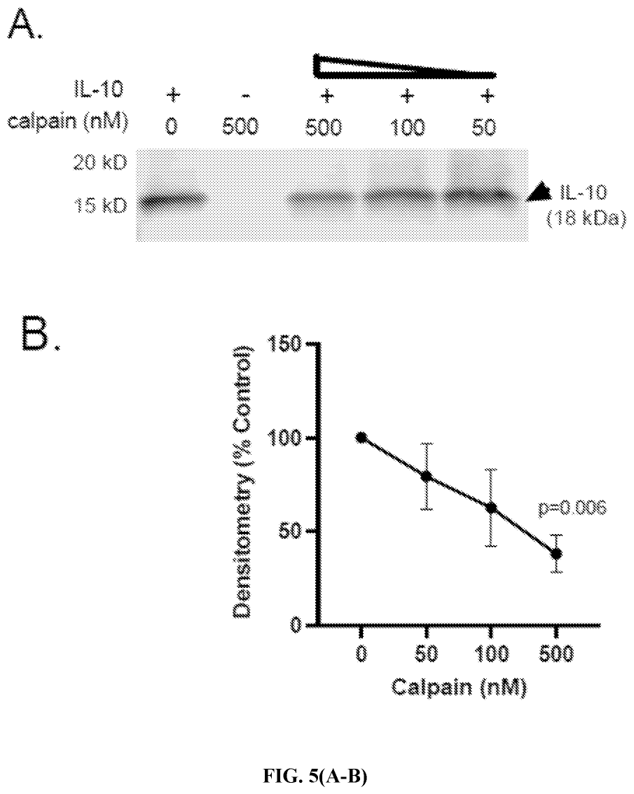

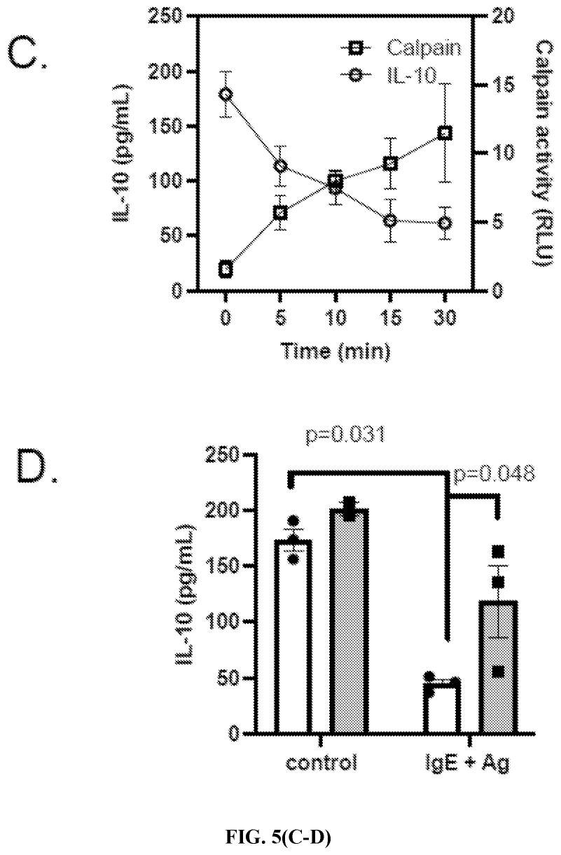

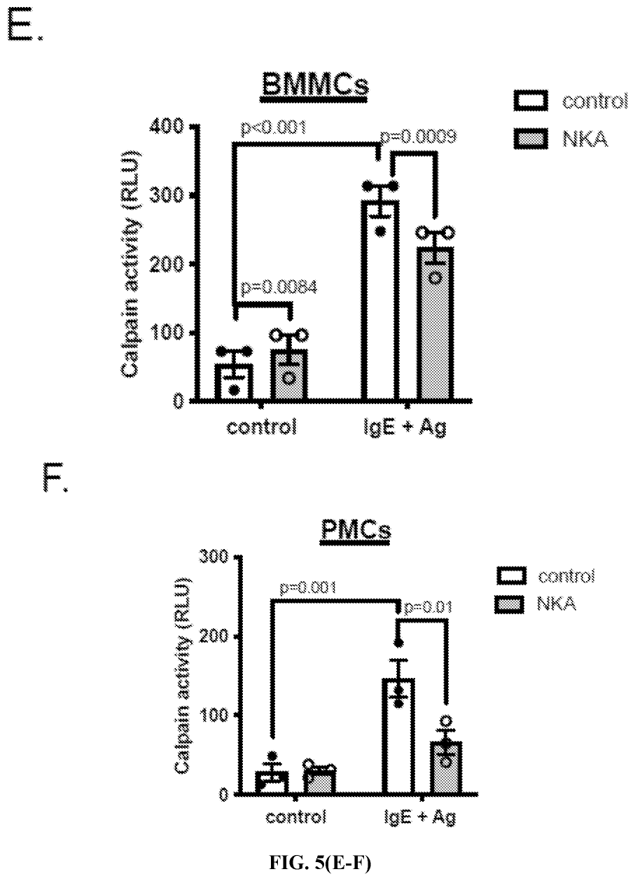

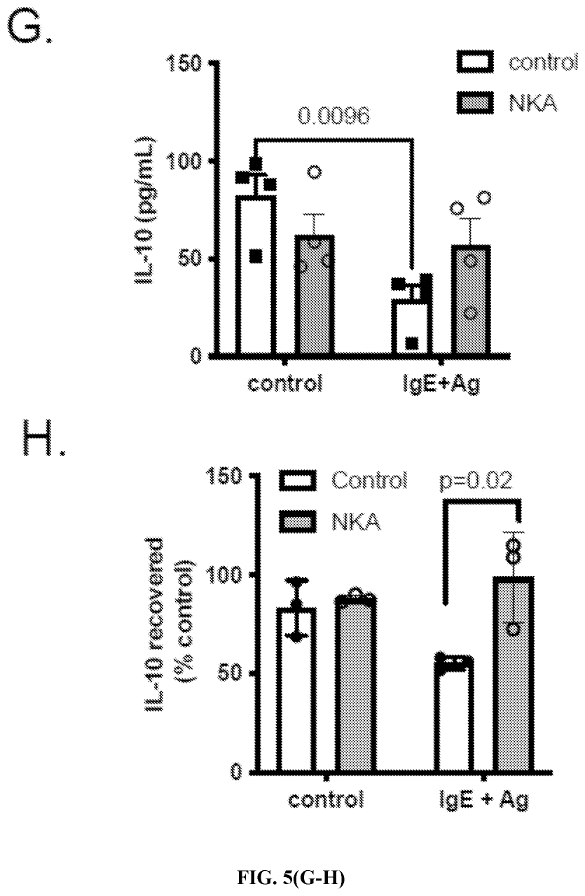

The objective of the proteomics screen was to identify a candidate molecule(s) released by FcεRI-activated MCs that was regulated by NKA. This widescreen suggested that extracellular calpain may be regulated by NKA. This observation was confirmed, evaluating calpain activity in cell free supernatants following IgE activation. Activating MCs led to an increase in extracellular calpain activity from BMMCs that was significantly downregulated by NKA (FIG. 5A). Importantly, calpain activity was also detected in PMCs supernatants and was significantly diminished by NKA (FIG. 5B). While NKA had diverging effects the conventional markers of exocytosis when evaluated in BMMCs and PMCs, it effected the capacity to downregulate calpain release similarly.

### Example 11. Neurokinin a Regulates Exteriorization of the IL-10 Degrading Cysteine Protease, Calpain

In MCs, intracellular calpain is required for IgE-initiated degranulation, NFκB activation, downstream cytokine production. But, the role of extracellular calpain in MC biology has not been explored, though it has been reported in a similar proteomics study evaluating the MC secretome of BMMCs and PMCs in response to FcεRI ligation.

The capacity for NKA to regulate IgE-initiated MC responses in vitro and in vivo required autocrine IL-10. Interestingly, IL-10 is degraded by cysteine proteases, such as calpain and bioinformatic analysis confirmed calpain cleavage sites in the mouse IL-10 amino acid sequence (data not shown). To directly test the relationship between calpain and IL-10, recombinant (r) IL-10 was incubated with increasing concentrations of purified calpain then IL-10 was detected by Western blotting. In accord with bioinformatic predictions, the amount of IL-10 detected by Western blot was inversely proportional to calpain concentration (FIGS. 5C and D).

The early kinetics of IL-10 release from IgE-activated MCs were investigated in relation to calpain activity. BMMCs that had rested for a minimum of 3 h in serum-free media prior to activation were used. This rest period allowed for MC-released IL-10 to accumulate. Within 5 min of activation, IL-10 levels decreased. Conversely, calpain activity increased (FIG. 5E). The hypothesis that the reduction in IL-10 following activation was related to exteriorized calpain was then tested. Intracellular calpain activity is necessary for MC degranulation, limiting approaches for directly investigating MC-exteriorized calpain and IL-10 degradation. To overcome this limitation, cell-free supernatants were collected from BMMCs 10 min after activation and pulsed them with a calpain inhibitor, Ac-calpastatin, for 30 min. Ac-calpastatin inhibited calpain activity 20.1±1.72% (not shown, mean±SEM from n=3). Pulsing supernatants from IgE-activated BMMCs with Ac-calpastatin partially restored IL-10 levels (FIG. 5F), demonstrating that calpain degraded autocrine IL-10. Collectively, these data show that IgE-activated MCs release calpain and that this calpain degrades extracellular IL-10.

In the steady state, MCs release low levels of IL-10, which diminished after IgE-activation in a calpain-dependent manner. NKA inhibits calpain release, so the relationship between NKA and early IL-10 levels was evaluated. Supernatants from BMMCs activated with NKA contained significantly more IL-10, suggesting that NKA inhibited early proteolytic degradation of IL-10 (FIG. 5G). To more specifically test this, BMMCs were activated with and without NKA, and cell-free supernatants were collected then pulsed with rIL-10 for 30 min and quantified by ELISA. Significantly less IL-10 was recovered from cell-free supernatants of BMMCs activated with IgE+Ag when compared to the amount of IL-10 detectable in control media alone or in ‘degranulation supernatants’ collected from either untreated or NKA-treated BMMCs (FIG. 5H). This data is consistent with the hypothesis that BMMCs release proteases, including calpain, which degrade early IL-10 and that this pathway is disrupted by NKA.

### Example 12. NKA Down-Regulates Surface Expression of the Calpain-Releasing ABCA1

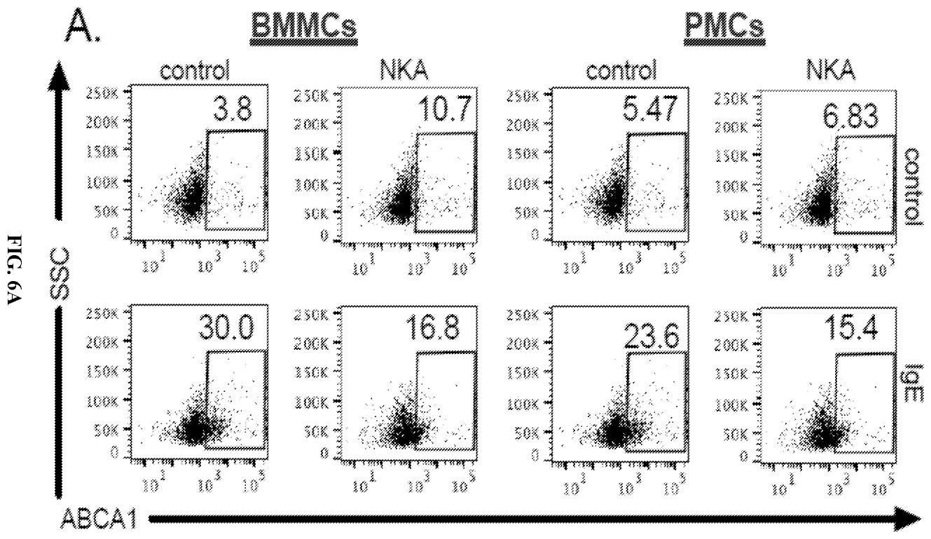

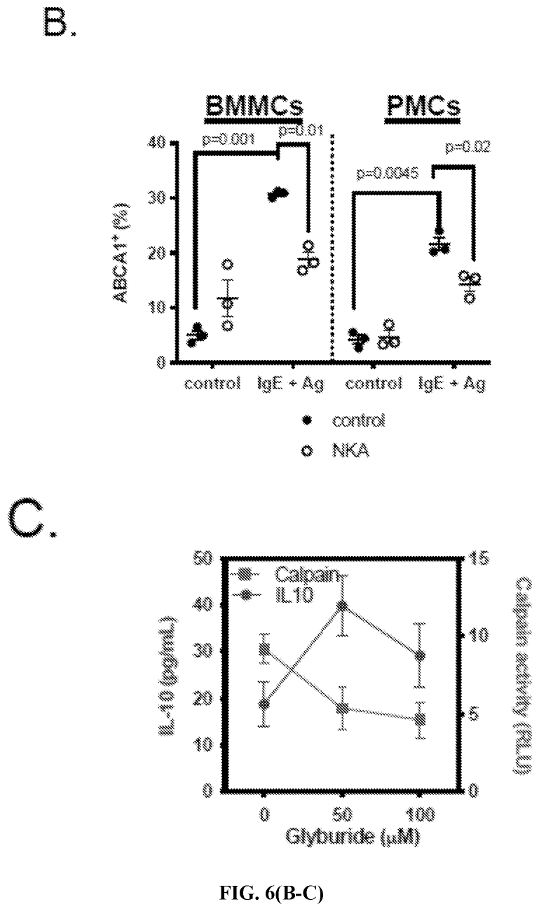

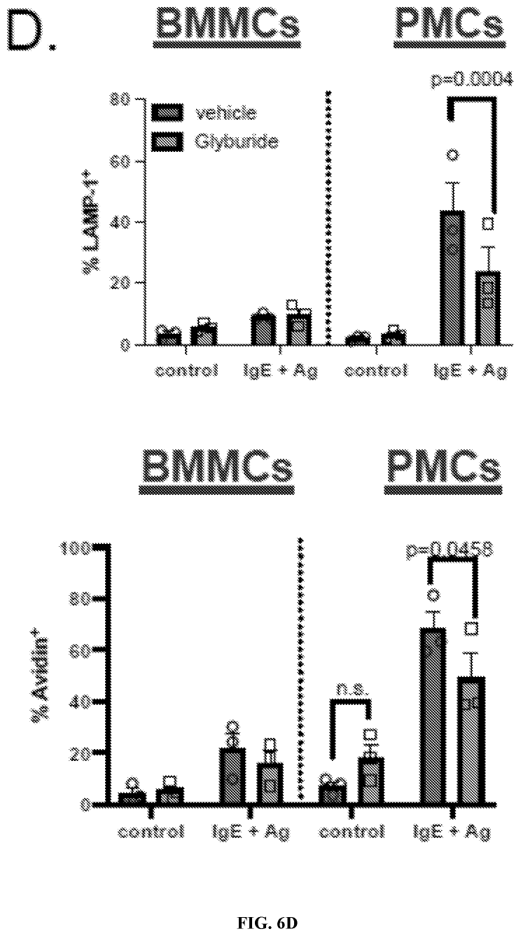

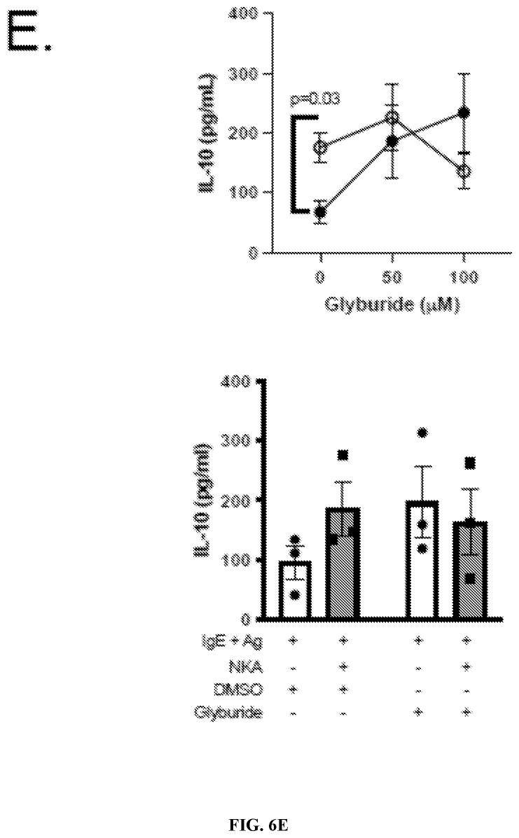

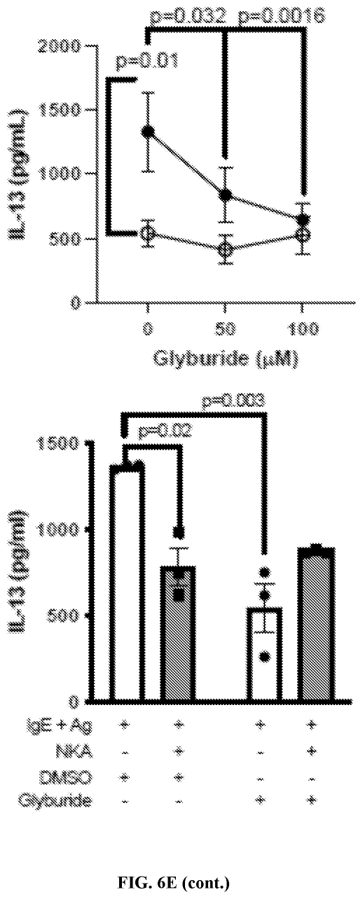

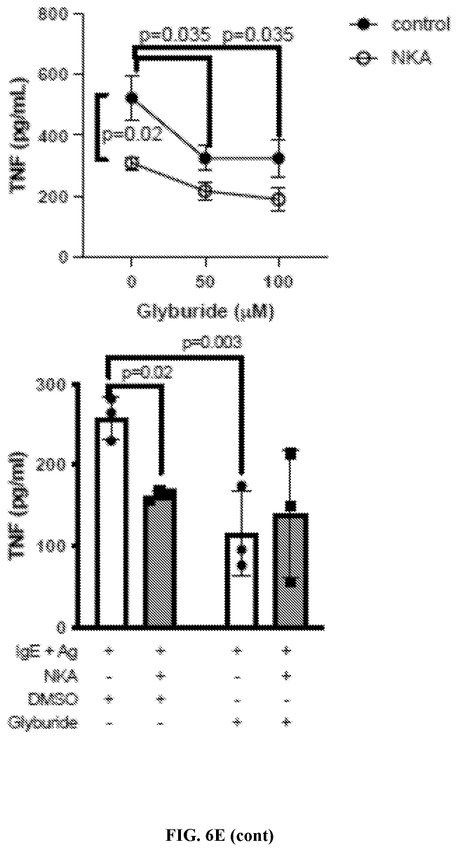

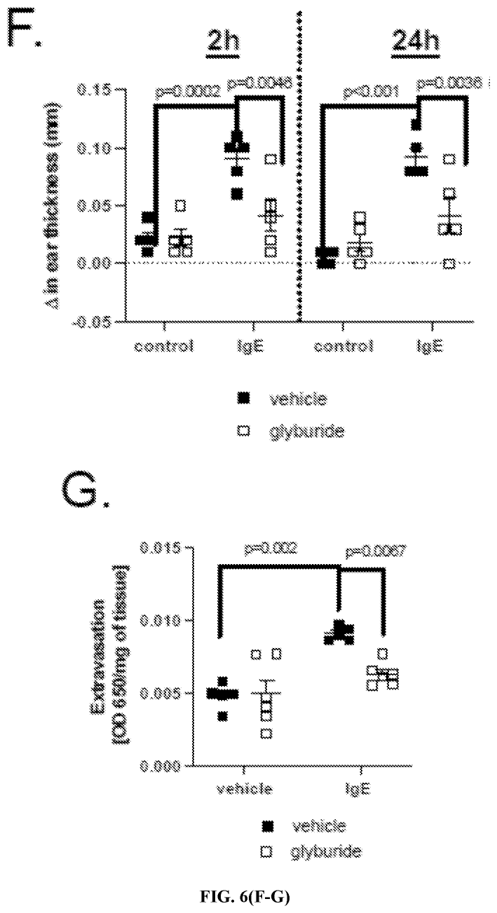

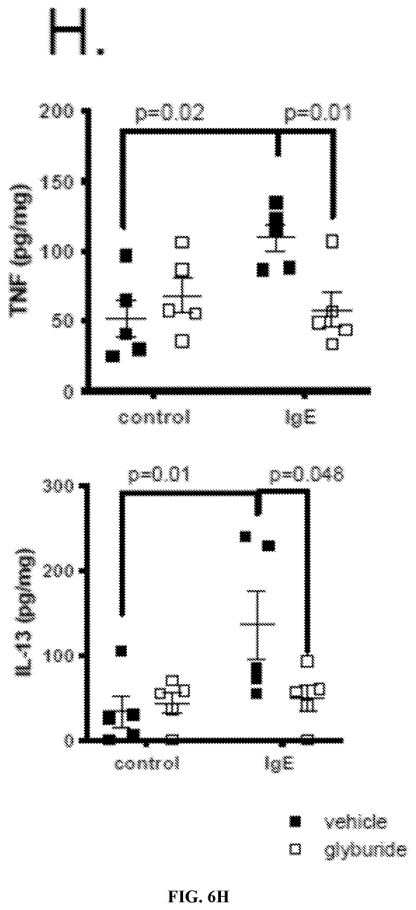

Calpain lacks a secretory signal and is exteriorized from T cells and melanomas through an exocytosis independent means involving the glyburide-sensitive ATP Binding Cassette (ABC) A1 channel. Surface ABCA1 expression on MCs activated with IgE+Ag in the presence or absence of NKA was investigated. Following IgE-initiated activation, ABCA1 expression was upregulated on both BMMCs and PMCs. The presence of NKA reduced the level of ABCA1 detected on the cell surface (FIG. 6A, B). It was then confirmed that calpain is released through the ABCA1 in MCs, treating BMMCs with increasing concentrations of glyburide and measuring calpain activity and IL-10 levels after activation. Glyburide reduced calpain activity while increasing IL-10 levels (FIG. 6C), suggesting that in MCs, calpain is released through the ABCA1 as has been reported in other cells. Collectively, these data demonstrate that MCs release IL-10 degrading calpain through the ABCA1 channel and that this pathway is modulated by NKA.

### Example 13. Inhibition of the ABCA1 Blocks MC Activation In Vitro and In Vivo

Neurokinin A exclusively inhibited exocytosis and the release of granulated proteins in PMCs, but not in BMMCs. Like NKA, glyburide reduced the percentage of LAMP-1⁺ and avidin⁺ PMCs after IgE-initiated activation, while having a less robust effect on BMMCs (FIG. 6D). Neurokinin A blocked IgE-induced IL-13 and TNF release as well as ABCA1 surface expression. It was hypothesized that inhibition of ABCA1 function with glyburide would pheno-copy the effects of NKA. After 20-24 h in culture, glyburide increased the detectible levels of IL-10 and decreased levels of IL-13 and TNF mimicking the effects of NKA in both BMMCs and PMCs (FIG. 6E). Importantly, glyburide did not further increase NKA-mediated inhibition, suggesting that NKA and ABCA1 inhibition are functionally redundant. It was then hypothesized that inhibition of the ABCA1 prior to PCA induction would be regulatory, paralleling the effects of NKA. To test this, mice were treated with glyburide 3 h prior to PCA induction. Glyburide significantly reduced ear thickness at 2 h and 24 h and diminished levels of IL-13 and TNF in the skin (FIG. 6F-H). Collectively, the data suggest that NKA reduces ABCA1 expression, and that blocking ABCA1 inhibits FcεRI-initiated MC activation.

### Example 14. Discussion

Neuropeptides initiate and amplify cutaneous inflammation and have also been shown to regulate allergic immune responses. Accordingly, NKA impedes MC activation in an IL-10 dependent manner, decreasing ABCA1-dependent release of the IL-10-degrading enzyme calpain and increasing IL10 transcription. Through multi-tiered IL-10 regulation, NKA prolongs the availability of IL-10 in the microenvironment and reduces MC function in vitro and in vivo.

The role of IL-10 in MC-mediated immune responses differs by experimental model. In vitro, autocrine IL-10 inhibits IgE-initiated MC activation and prolonged IL-10 treatment inhibits FcεRI expression, STAT5 activation and TNF release initiated by IgE but selectively enhances IL-13 release. In contrast to this, IL-10-deficient BMMCs have impaired TNF and IL-13 release and the absence of IL-10 does not affect degranulation. In line with these observations, it is shown herein using two different systems that PMCs and BMMCs lacking autocrine IL-10 have impaired IgE-initiated responses. However, the capacity for NKA to suppress MC function relied on IL-10 in vitro. Collectively, these studies suggest that autocrine IL-10 may be necessary for in vitro differentiation of immuno-active MCs, but that, in the course of FcεRI-initiated activation, autocine IL-10 may be inhibitory.

In vivo, the functions of IL-10 in MC biology also diverge. In mucosal tissues, IL-10 enhances IgE-initiated MC activation. But, in the skin, MC-derived IL-10 is associated with reduced inflammation in models of UV-induced immune suppression, contact dermatitis and CHS. In our hands, the absence of autocrine IL-10 did not affect the early phase of PCA, but TNF levels were increased in the late phase of PCA. Importantly, the capacity for NKA to downregulate PCA required IL-10. Collectively, these data suggest that the relationship between MCs and IL-10 in vivo may depend on the tissue and that, in the skin, IL-10 regulates MC function.

Previous studies investigating NKA or a naturally truncated form of NKA (NKA(4-10)) on MCs have shown no effect. These studies relied on β-hexosaminidase or histamine release as readouts for MC activation. In accord with these studies, no effect for NKA on β-hexosaminidase release from BMMCs or PMCs was found. However, when the study was extended to include other hallmarks of early MC activation, it was found that NKA blocked exteriorization of avidin⁺ granules, but only in PMCs. Both BMMCs and PMCs express equivalent levels of the NK2R, ruling out receptor availability as a factor in this difference. PMCs are fundamentally more mature than BMMCs and signaling pathways associated with maturation may be important in considering this difference. This finding highlights the importance of using multiple types of MCs when evaluating early events downstream of activation.

The total secretome was screened to identify proteins that may be affected by NKA outside of those classically evaluated in MCs. This approach revealed differential protein release from MCs in the presence of NKA including calpain I. Importantly, calpain activity was readily detectible in supernatants from both IgE-activated BMMCs and PMCs and the release of calpain through the ABCA1 may be a common mechanism utilized by different types of MCs, regardless of maturation state to regulate the extracellular environment.

The availability of calpain substrates in the extracellular environment may increase inflammation and broadly impact the outcome of immune responses. An inverse relationship between calpain activity and IL-10 in MC supernatants is demonstrated here. A similar relationship has been noted in the peripheral blood mononuclear cells from MS patients and in a rodent model of traumatic brain injury. Other studies have shown that IL-10 is highly susceptible to degradation by cysteine proteases such as calpain, and pulsing T cell supernatants with calpain diminishes IL-10 levels. Building on these findings, it is directly demonstrated that calpain degrades IL-10. In this study, calpain activity and subsequent IL-10 degradation link early events following MC activation with later release of inflammatory mediators providing support for the hypothesis that exteriorized calpain promotes inflammation.

NKA was utilized to uncover a novel mode of protease release from MCs via the glyburide-sensitive ABCA1 channel. ABCA1 levels increased on the cell surface of MCs following IgE-initiated activation. This may reflect convergence of multiple pathways downstream of the FcεRI, including cAMP/PKA and PKC. Elucidation of the exact intracellular mechanism linking the NKA and NK2R signaling to ABCA1 at the cell surface is beyond the scope of the present study. But, GPCRs such as the NK2R interact via specific Ga subunits to either activate or regulate PKA. Downstream ABCA1 regulation may be a common mechanism utilized by GPCRs to control the MC secretome.

The ABCA1 is best characterized as a lipid transporter but immune functions related to ABCA1 activity have been described. Inhibition of ABCA1 activity with glyburide inhibits the inflammasome, transcription of inflammatory mediators, including TNF and is anti-inflammatory and protective in diabetic patients in sepsis. Studies evaluating ABCA1 activity in MCs are limited but suggest that translocation of the ABCA1 to the cell surface may be important for degranulation and early cytokine release. It is demonstrated here that ABCA1 inhibition mirrors the effects of NKA in vitro and in vivo. ABCA1 activity was associated with reduced IL-10, increased TNF and IL-13 in vitro and increased MC responses in PCA. Collectively, ABCA1 activation is associated with inflammation and local manipulation of this pathway may prove to be clinically important.

Neurokinin A is released from TRPV1⁺ sensory nerve fibers and inhibits MC responses through IL-10 and calpain regulation. In the steady state, NKA levels are low in mouse skin, and upregulated following MC activation in IL-10-dependent fashion. This feedback loop may ultimately dampen inflammation but also dampen the response of peripheral sensory nerve fibers. Dorsal root ganglion (DRG) express the IL-10R and IL-10 dampens neuropathic pain and reduces thermal hyposensitivity. Further, DRG directly respond to cysteine proteases. In addition to downregulating MC responses, the NKA-calpain-IL10 pathway may temper nociceptor activation within the MC-neuron synapse. Exploitation of this pathway may open new therapeutic strategies targeting both MCs and neurons to reduce allergic inflammation.

### REFERENCES

1. Cheng L E, Hartmann K, Roers A, Krummel M F, Locksley R M. Perivascular mast cells dynamically probe cutaneous blood vessels to capture immunoglobulin E. Immunity 2013; 38:166-75.
2. Klein O, Sagi-Eisenberg R. Anaphylactic Degranulation of Mast Cells: Focus on Compound Exocytosis. J Immunol Res 2019; 2019:9542656.
3. Mitra P, Oskeritzian C A, Payne S G, Beaven M A, Milstien S, Spiegel S. Role of ABCC1 in export of sphingosine-1-phosphate from mast cells. Proc Natl Acad Sci USA 2006; 103:16394-9.
4. Taracanova A, Tsilioni I, Conti P, Norwitz E R, Leeman S E, Theoharides T C. Substance P and IL-33 administered together stimulate a marked secretion of IL-1beta from human mast cells, inhibited by methoxyluteolin. Proc Natl Acad Sci USA 2018; 115:E9381-E90.
5. Tanaka N, Kawai J, Hirasawa N, Mano N, Yamaguchi H. ATP-Binding Cassette Transporter C4 is a Prostaglandin D2 Exporter in HMC-1 cells. Prostaglandins Leukot Essent Fatty Acids 2020; 159:102139.
6. Pullen N A, Falanga Y T, Morales J K, Ryan J J. The Fyn-STAT5 Pathway: A New Frontier in IgE- and IgG-Mediated Mast Cell Signaling. Front Immunol 2012; 3:117.
7. Kitaura J, Asai K, Maeda-Yamamoto M, Kawakami Y, Kikkawa U, Kawakami T. Akt-dependent cytokine production in mast cells. J Exp Med 2000; 192:729-40.
8. Starkl P, Watzenboeck M L, Popov L M, Zahalka S, Hladik A, Lakovits K, et al. IgE Effector Mechanisms, in Concert with Mast Cells, Contribute to Acquired Host Defense against Staphylococcusaureus. Immunity 2020; 53:793-804 e9.
9. Alving K, Sundstrom C, Matran R, Panula P, Hokfelt T, Lundberg J M. Association between histamine-containing mast cells and sensory nerves in the skin and airways of control and capsaicin-treated pigs. Cell Tissue Res 1991; 264:529-38.
10. Undem B J, Taylor-Clark T. Mechanisms underlying the neuronal-based symptoms of allergy. J Allergy Clin Immunol 2014; 133:1521-34.
11. Kakurai M, Monteforte R, Suto H, Tsai M, Nakae S, Galli S J. Mast cell-derived tumor necrosis factor can promote nerve fiber elongation in the skin during contact hypersensitivity in mice. Am J Pathol 2006; 169:1713-21.
12. Dalsgaard C J, Haegerstrand A, Theodorsson-Norheim E, Brodin E, Hokfelt T.

Neurokinin A-like immunoreactivity in rat primary sensory neurons; coexistence with substance P. Histochemistry 1985; 83:37-9.
13. Bakhle Y S, Bell C. Neurokinin A and substance P vary independently in different regions of rat sensory neurons. Neuropeptides 1995; 28:237-41.
14. Schulze E, Witt M, Fink T, Hofer A, Funk R H. Immunohistochemical detection of human skin nerve fibers. Acta Histochem 1997; 99:301-9.
15. Eedy D J, Shaw C, Johnston C F, Buchanan K D. The regional distribution of neuropeptides in human skin as assessed by radioimmunoassay and high-performance liquid chromatography. Clin Exp Dermatol 1994; 19:463-72.
16. Gaudenzio N, Sibilano R, Marichal T, Starkl P, Reber L L, Cenac N, et al. Different activation signals induce distinct mast cell degranulation strategies. J Clin Invest 2016; 126:3981-98.
17. Sumpter T L, Ho C H, Pleet A R, Tkacheva O A, Shufesky W J, Rojas-Canales D M, et al. Autocrine hemokinin-1 functions as an endogenous adjuvant for IgE-mediated mast cell inflammatory responses. The Journal of allergy and clinical immunology 2014.
18. Choi H, Kim D J, Nam S, Lim S, Hwang J S, Park K S, et al. Manifestation of atopic dermatitis-like skin in TNCB-induced N C/Nga mice is ameliorated by topical treatment of substance P, possibly through blockade of allergic inflammation. Exp Dermatol 2018; 27:396-402.
19. Steinhoff M S, von Mentzer B, Geppetti P, Pothoulakis C, Bunnett N W. Tachykinins and their receptors: contributions to physiological control and the mechanisms of disease. Physiol Rev 2014; 94:265-301.
20. Schuiling M, Zuidhof A B, Meurs H, Zaagsma J. Role of tachykinin NK2-receptor activation in the allergen-induced late asthmatic reaction, airway hyperreactivity and airway inflammatory cell influx in conscious, unrestrained guinea-pigs. Br J Pharmacol 1999; 127:1030-8.
21. Delvalle N M, Dharshika C, Morales-Soto W, Fried D E, Gaudette L, Gulbransen B D. Communication Between Enteric Neurons, Glia, and Nociceptors Underlies the Effects of Tachykinins on Neuroinflammation. Cell Mol Gastroenterol Hepatol 2018; 6:321-44.
22. Scholzen T E, Steinhoff M, Sindrilaru A, Schwarz A, Bunnett N W, Luger T A, et al. Cutaneous allergic contact dermatitis responses are diminished in mice deficient in neurokinin 1 receptors and augmented by neurokinin 2 receptor blockage. FASEB J 2004; 18:1007-9.
23. Cross L J, Heaney L G, Ennis M. Histamine release from human bronchoalveolar lavage mast cells by neurokinin A and bradykinin. Inflamm Res 1997; 46:306-9.
24. Lowman M A, Benyon R C, Church M K. Characterization of neuropeptide-induced histamine release from human dispersed skin mast cells. Br J Pharmacol 1988; 95:121-30.
25. Bischoff S C, Schwengberg S, Lorentz A, Manns M P, Bektas H, Sann H, et al. Substance P and other neuropeptides do not induce mediator release in isolated human intestinal mast cells. Neurogastroenterol Motil 2004; 16:185-93.
26. Levick S P, Brower G L, Janicki J S. Substance β-mediated cardiac mast cell activation: An in vitro study. Neuropeptides 2019; 74:52-9.
27. Melendez G C, Li J, Law B A, Janicki J S, Supowit S C, Levick S P. Substance P induces adverse myocardial remodelling via a mechanism involving cardiac mast cells. Cardiovasc Res 2011; 92:420-9.
28. Mukai H, Kikuchi M, Fukuhara S, Kiso Y, Munekata E. Cryptide signaling: Amphiphilic peptide-induced exocytotic mechanisms in mast cells. Biochem Biophys Res Commun 2008; 375:22-6.
29. Roers A, Siewe L, Strittmatter E, Deckert M, Schluter D, Stenzel W, et al. T cell-specific inactivation of the interleukin 10 gene in mice results in enhanced T cell responses but normal innate responses to lipopolysaccharide or skin irritation. J Exp Med 2004; 200:1289-97.
30. Reber L L, Sibilano R, Starkl P, Roers A, Grimbaldeston M A, Tsai M, et al. Imaging protective mast cells in living mice during severe contact hypersensitivity. JCI Insight 2017; 2.
31. Jensen B M, Swindle E J, Iwaki S, Gilfillan A M. Generation, isolation, and maintenance of rodent mast cells and mast cell lines. Curr Protoc Immunol 2006; Chapter 3:Unit 3 23.
32. Krutzik P O, Nolan G P. Intracellular phospho-protein staining techniques for flow cytometry: monitoring single cell signaling events. Cytometry A 2003; 55:61-70.
33. Maguire O, Collins C, O'Loughlin K, Miecznikowski J, Minderman H. Quantifying nuclear p65 as a parameter for NF-kappaB activation: Correlation between ImageStream cytometry, microscopy, and Western blot. Cytometry A 2011; 79:461-9.
34. Wisniewski J R, Zougman A, Nagaraj N, Mann M. Universal sample preparation method for proteome analysis. Nat Methods 2009; 6:359-62.
35. Perez-Riverol Y, Csordas A, Bai J, Bernal-Llinares M, Hewapathirana S, Kundu D J, et al. The PRIDE database and related tools and resources in 2019: improving support for quantification data. Nucleic Acids Res 2019; 47:D442-D50.
36. Deutsch E W, Bandeira N, Sharma V, Perez-Riverol Y, Carver J J, Kundu D J, et al. The ProteomeXchange consortium in 2020: enabling ‘big data’ approaches in proteomics. Nucleic Acids Res 2020; 48:D1145-D52.
37. Kitamura H, Kobayashi M, Wakita D, Nishimura T. Neuropeptide signaling activates dendritic cell-mediated type 1 immune responses through neurokinin-2 receptor. J Immunol 2012; 188:4200-8.
38. Malbec O, Roget K, Schiffer C, Iannascoli B, Dumas A R, Arock M, et al. Peritoneal cell-derived mast cells: an in vitro model of mature serosal-type mouse mast cells. J Immunol 2007; 178:6465-75.
39. Meurer S K, Ness M, Weiskirchen S, Kim P, Tag C G, Kauffmann M, et al. Isolation of Mature (Peritoneum-Derived) Mast Cells and Immature (Bone Marrow-Derived) Mast Cell Precursors from Mice. PLoS One 2016; 11:e0158104.
40. Ando T, Xiao W, Gao P, Namiranian S, Matsumoto K, Tomimori Y, et al. Critical role for mast cell Stat5 activity in skin inflammation. Cell Rep 2014; 6:366-76.
41. Pullen N A, Barnstein B O, Falanga Y T, Wang Z, Suzuki R, Tamang T D, et al. Novel mechanism for Fc{epsilon}R I-mediated signal transducer and activator of transcription 5 (STAT5) tyrosine phosphorylation and the selective influence of STAT5B over mast cell cytokine production. J Biol Chem 2012; 287:2045-54.
42. Barnstein B O, Li G, Wang Z, Kennedy S, Chalfant C, Nakajima H, et al. Stat5 expression is required for IgE-mediated mast cell function. J Immunol 2006; 177:3421-6.
43. Royer B, Varadaradjalou S, Saas P, Gabiot A C, Kantelip B, Feger F, et al. Autocrine regulation of cord blood-derived human mast cell activation by IL-10. J Allergy Clin Immunol 2001; 108:80-6.
44. Kennedy Norton S, Barnstein B, Brenzovich J, Bailey D P, Kashyap M, Speiran K, et al. IL-10 suppresses mast cell IgE receptor expression and signaling in vitro and in vivo. J Immunol 2008; 180:2848-54.
45. Polukort S H, Rovatti J, Carlson L, Thompson C, Ser-Dolansky J, Kinney S R, et al. IL-10 Enhances IgE-Mediated Mast Cell Responses and Is Essential for the Development of Experimental Food Allergy in IL-10-Deficient Mice. J Immunol 2016; 196:4865-76.
46. Madan R, Demircik F, Surianarayanan S, Allen J L, Divanovic S, Trompette A, et al. Nonredundant roles for B cell-derived IL-10 in immune counter-regulation. J Immunol 2009; 183:2312-20.
47. Joulia R, Gaudenzio N, Rodrigues M, Lopez J, Blanchard N, Valitutti S, et al. Mast cells form antibody-dependent degranulatory synapse for dedicated secretion and defence. Nat Commun 2015; 6:6174.
48. Moon T C, Befus A D, Kulka M. Mast cell mediators: their differential release and the secretory pathways involved. Front Immunol 2014; 5:569.
49. Grutzkau A, Smorodchenko A, Lippert U, Kirchhof L, Artuc M, Henz B M. LAMP-1 and LAMP-2, but not LAMP-3, are reliable markers for activation-induced secretion of human mast cells. Cytometry A 2004; 61:62-8.
50. Zhang B, Asadi S, Weng Z, Sismanopoulos N, Theoharides T C. Stimulated human mast cells secrete mitochondrial components that have autocrine and paracrine inflammatory actions. PLoS One 2012; 7:e49767.
51. Wu Z, Chen X, Liu F, Chen W, Wu P, Wieschhaus A J, et al. Calpain-1 contributes to IgE-mediated mast cell activation. J Immunol 2014; 192:5130-9.
52. Shubin N J, Glukhova V A, Clauson M, Truong P, Abrink M, Pejler G, et al. Proteome analysis of mast cell releasates reveals a role for chymase in the regulation of coagulation factor XIIIA levels via proteolytic degradation. J Allergy Clin Immunol 2017; 139:323-34.
53. Duwadi K, Chen L, Menassa R, Dhaubhadel S. Identification, Characterization and Down-Regulation of Cysteine Protease Genes in Tobacco for Use in Recombinant Protein Production. PLoS One 2015; 10:e0130556.
54. Perez J, Dansou B, Herve R, Levi C, Tamouza H, Vandermeersch S, et al. Calpains Released by T Lymphocytes Cleave TLR2 To Control IL-17 Expression. J Immunol 2016; 196:168-81.
55. Hanouna G, Tang E, Perez J, Vandermeersch S, Haymann J P, Baud L, et al. Preventing Calpain Externalization by Reducing ABCA1 Activity with Probenecid Limits Melanoma Angiogenesis and Development. J Invest Dermatol 2020; 140:445-54.
56. Siebenhaar F, Magerl M, Peters E M, Hendrix S, Metz M, Maurer M. Mast cell-driven skin inflammation is impaired in the absence of sensory nerves. J Allergy Clin Immunol 2008; 121:955-61.
57. Riol-Blanco L, Ordovas-Montanes J, Perro M, Naval E, Thiriot A, Alvarez D, et al. Nociceptive sensory neurons drive interleukin-23-mediated psoriasiform skin inflammation. Nature 2014; 510:157-61.
58. Cohen J A, Edwards T N, Liu A W, Hirai T, Jones M R, Wu J, et al. Cutaneous TRPV1(+) Neurons Trigger Protective Innate Type 17 Anticipatory Immunity. Cell 2019; 178:919-32 e14.
59. Serhan N, Basso L, Sibilano R, Petitfils C, Meixiong J, Bonnart C, et al. House dust mites activate nociceptor-mast cell clusters to drive type 2 skin inflammation. Nat Immunol 2019; 20:1435-43.
60. Nagashima H, Mahlakoiv T, Shih H Y, Davis F P, Meylan F, Huang Y, et al. Neuropeptide CGRP Limits Group 2 Innate Lymphoid Cell Responses and Constrains Type 2 Inflammation. Immunity 2019; 51:682-95 e6.
61. Inclan-Rico J M, Ponessa J J, Valero-Pacheco N, Hernandez C M, Sy C B, Lemenze A D, et al. Basophils prime group 2 innate lymphoid cells for neuropeptide-mediated inhibition. Nat Immunol 2020; 21:1181-93.
62. Gillespie S R, DeMartino R R, Zhu J, Chong H J, Ramirez C, Shelburne C P, et al. IL-10 inhibits Fc epsilon R I expression in mouse mast cells. J Immunol 2004; 172:3181-8.
63. Qayum A A, Paranjape A, Abebayehu D, Kolawole E M, Haque T T, McLeod J J, et al. I L-10-Induced miR-155 Targets SOCS1 To Enhance IgE-Mediated Mast Cell Function. J Immunol 2016; 196:4457-67.
64. Grimbaldeston M A, Nakae S, Kalesnikoff J, Tsai M, Galli S J. Mast cell-derived interleukin 10 limits skin pathology in contact dermatitis and chronic irradiation with ultraviolet B. Nat Immunol 2007; 8:1095-104.
65. Gross O, Yazdi A S, Thomas C J, Masin M, Heinz L X, Guarda G, et al. Inflammasome activators induce interleukin-lalpha secretion via distinct pathways with differential requirement for the protease function of caspase-1. Immunity 2012; 36:388-400.
66. Hayakawa M, Hayakawa H, Matsuyama Y, Tamemoto H, Okazaki H, Tominaga S. Mature interleukin-33 is produced by calpain-mediated cleavage in vivo. Biochem Biophys Res Commun 2009; 387:218-22.
67. Valimaki E, Cypryk W, Virkanen J, Nurmi K, Turunen P M, Eklund K K, et al. Calpain Activity Is Essential for ATP-Driven Unconventional Vesicle-Mediated Protein Secretion and Inflammasome Activation in Human Macrophages. J Immunol 2016; 197:3315-25.
68. Imam S A, Guyton M K, Haque A, Vandenbark A, Tyor W R, Ray S K, et al. Increased calpain correlates with Th1 cytokine profile in PBMCs from M S patients. J Neuroimmunol 2007; 190:139-45.
69. Hu J, Chen L, Huang X, Wu K, Ding S, Wang W, et al. Calpain inhibitor MDL28170 improves the transplantation-mediated therapeutic effect of bone marrow-derived mesenchymal stem cells following traumatic brain injury. Stem Cell Res Ther 2019; 10:96.
70. Liu Y, Tang C. Regulation of ABCA1 functions by signaling pathways. Biochim Biophys Acta 2012; 1821:522-9.
71. Chen Y, Granger A J, Tran T, Saulnier J L, Kirkwood A, Sabatini B L. Endogenous Galphaq-Coupled Neuromodulator Receptors Activate Protein Kinase A. Neuron 2017; 96:1070-83 e5.
72. Lowes D J, Hevener K E, Peters B M. Second-Generation Antidiabetic Sulfonylureas Inhibit Candida albicans and Candidalysin-Mediated Activation of the NLRP3 Inflammasome. Antimicrob Agents Chemother 2020; 64.
73. Zhang G, Lin X, Zhang S, Xiu H, Pan C, Cui W. A Protective Role of Glibenclamide in Inflammation-Associated Injury. Mediators Inflamm 2017; 2017:3578702.
74. Koh G C, Maude R R, Schreiber M F, Limmathurotsakul D, Wiersinga W J, Wuthiekanun V, et al. Glyburide is anti-inflammatory and associated with reduced mortality in melioidosis. Clin Infect Dis 2011; 52:717-25.
75. Hagemann P M, Nsiah-Dosu S, Hundt J E, Hartmann K, Orinska Z. Modulation of Mast Cell Reactivity by Lipids: The Neglected Side of Allergic Diseases. Front Immunol 2019; 10:1174.
76. Strobel W M, Luscher T F, Simper D, Linder L, Haefeli W E. Substance P in human hand veins in vivo: tolerance, efficacy, potency, and mechanism of venodilator action. Clin Pharmacol Ther 1996; 60:435-43.
77. Schmid E, Leierer J, Doblinger A, Laslop A, Fischer-Colbrie R, Humpel C, et al.

Neurokinin a is a main constituent of sensory neurons innervating the anterior segment of the eye. Invest Ophthalmol Vis Sci 2005; 46:268-74.
78. Laumet G, Bavencoffe A, Edralin J D, Huo X J, Walters E T, Dantzer R, et al. Interleukin-10 resolves pain hypersensitivity induced by cisplatin by reversing sensory neuron hyperexcitability. Pain 2020.
79. Ren K, Dubner R. Interactions between the immune and nervous systems in pain. Nat Med 2010; 16:1267-76.
80. Stein C, Machelska H. Modulation of peripheral sensory neurons by the immune system: implications for pain therapy. Pharmacol Rev 2011; 63:860-81.
81. Wagner R, Janjigian M, Myers R R. Anti-inflammatory interleukin-10 therapy in CCI neuropathy decreases thermal hyperalgesia, macrophage recruitment, and endoneurial TNF-alpha expression. Pain 1998; 74:35-42.

1. A method of treating an inflammation in a subject comprising administering to the subject a pharmaceutically effective amount of a mast cell desensitizing composition.

2. A method of treating a type I hypersensitivity reaction in a subject comprising administering to the subject a pharmaceutically effective amount of a mast cell desensitizing composition.

3. The method of claim 2, wherein the type I hypersensitivity reaction is selected from the group consisting of asthma, rhinitis, conjunctivitis, and dermatitis.

4. The method of claim 3, wherein the dermatitis is psoriasis, eczema or seborrheic dermatitis.

5. The method of claim 4, wherein the eczema is atopic dermatitis.

6. The method of claim 2, wherein the type I hypersensitivity reaction is selected from the group consisting of anaphylaxis, urticaria, and angioedema.

7. The method of claim 1, further comprising administering one or more type I hypersensitivity reaction antigens to the subject.

8. The method of claim 7, wherein the one or more type I hypersensitivity reaction antigens are administered to a same cutaneous microenvironment as the mast cell desensitizing composition.

9. The method of claim 2, wherein the type I hypersensitivity reaction is a food allergy or a drug allergy.

10. The method of claim 9, wherein the type I hypersensitivity reaction is a peanut allergy and wherein the type I hypersensitivity reaction antigen is a peanut antigen.

11. The method of claim 1, wherein the mast cell is a connective tissue mast cell.

12. The method of claim 1, wherein the mast cell is a mucosal mast cell.

13. The method of claim 1, wherein the mast cell desensitizing composition comprises an ABCA1 inhibitor.

14. The method of claim 13, wherein the ABCA1 inhibitor is selected from the group of a glyburide, a probucol, a tocofersolan and a Vitamin E.

15. The method of claim 13, wherein the ABCA1 inhibitor is a glyburide.

16. The method of claim 1, wherein the mast cell desensitizing composition comprises a neurokinin A.

17. The method of claim 16, wherein the neurokinin A has a sequence of HKTDSFVGLM (SEQ ID NO:2) or a functional fragment thereof.

18. The method of claim 1, wherein the mast cell desensitizing composition is botulinum toxin A.

19. The method of claim 1, further comprising administering to the subject an IL-10.

20. The method of claim 1, wherein the administration is intradermal or transdermal.

21. The method of claim 20, wherein the administration is via one or more microneedles.

22. The method of claim 21, wherein the one or more microneedles are dissolvable.


SEQUENCE LISTING (12 sequences)

SEQ ID NO: 1 - protein, 2261 aa, Homo sapiens
MACWPQLRLLLWKNLTFRRRQTCQLLLEVAWPLFIFLILISVRLSYPPYEQHECHFPNKAMPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFASLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDAIKQMKKHLKLAKDSSADRFLNSLGRFMTGLDTKNNVKVWFNNKGWHAISSFLNVINNAILRANLQKGENPSHYGITAFNHPLNLTKQQLSEVALMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNKNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVLKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV

SEQ ID NO: 2 - protein, 10 aa, Homo sapiens
HKTDSFVGLM

SEQ ID NO: 3 - protein, 714 aa, Homo sapiens
MSEEIITPVYCTGVSAQVQKQRARELGLGRHENAIKYLGQDYEQLRVRCLQSGTLFRDEAFPPVPQSLGYKDLGPNSSKTYGIKWKRPTELLSNPQFIVDGATRTDICQGALGDCWLLAAIASLTLNDTLLHRVVPHGQSFQNGYAGIFHFQLWQFGEWVDVVVDDLLPIKDGKLVFVHSAEGNEFWSALLEKAYAKVNGSYEALSGGSTSEGFEDFTGGVTEWYELRKAPSDLYQIILKALERGSLLGCSIDISSVLDMEAITFKKLVKGHAYSVTGAKQVNYRGQVVSLIRMRNPWGEVEWTGAWSDSSSEWNNVDPYERDQLRVKMEDGEFWMSFRDFMREFTRLEICNLTPDALKSRTIRKWNTTLYEGTWRRGSTAGGCRNYPATFWVNPQFKIRLDETDDPDDYGDRESGCSFVLALMQKHRRRERRFGRDMETIGFAVYEVPPELVGQPAVHLKRDFFLANASRARSEQFINLREVSTRFRLPPGEYVVVPSTFEPNKEGDFVLRFFSEKSAGTVELDDQIQANLPDEQVLSEEEIDENFKALFRQLAGEDMEISVKELRTILNRIISKHKDLRTKGFSLESCRSMVNLMDRDGNGKLGLVEFNILWNRIRNYLSIFRKFDLDKSGSMSAYEMRMAIESAGFKLNKKLYELIITRYSEPDLAVDFDNFVCCLVRLETMFRFFKTLDTDLDGVVTFDLFKWLQLTMFA

SEQ ID NO: 4 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 5 - DNA, 20 bp, Artificial Sequence
  synthetic construct
ccgatgggttgtaccttgtc

SEQ ID NO: 6 - DNA, 20 bp, Artificial Sequence
  synthetic construct
cggactccgcaaagtctaag

SEQ ID NO: 7 - DNA, 19 bp, Artificial Sequence
  synthetic construct
cctggctcttgcttgcctt

SEQ ID NO: 8 - DNA, 21 bp, Artificial Sequence
  synthetic construct
ggtcttgtgtgatgttgctca

SEQ ID NO: 9 - DNA, 22 bp, Artificial Sequence
  synthetic construct
gctggacaacatactgctaacc

SEQ ID NO: 10 - DNA, 20 bp, Artificial Sequence
  synthetic construct
atttccgataagcttggcaa

SEQ ID NO: 11 - DNA, 20 bp, Artificial Sequence
  synthetic construct
ggctgtattcccctccatcg

SEQ ID NO: 12 - DNA, 22 bp, Artificial Sequence
  synthetic construct
ccagttggtaacaatgccatgt